(19) United States
(12) Patent Application Publication
(10) Pub. No.: US 2025/0162980 A1
(43) Pub. Date: May 22, 2025

(54) METHODS FOR PREPARING MESCALINE

(71) Applicant: Journey Colab Corp., San Francisco, CA (US)

(72) Inventors: Derek Londesbrough, Hartlepool (GB); Christopher Gill, Sedgefield (GB)

(21) Appl. No.: 18/841,469

(22) PCT Filed: Feb. 24, 2023
(86) PCT No.: PCT/US2023/013886

§ 371 Date: Aug. 26, 2024

Related U.S. Application Data
(60) Provisional application No. 63/314,129, filed on Feb. 25, 2022.
(60) Provisional application No. 63/345,602, filed on May 25, 2022.

(51) Int. Cl. C07C 217/60; A61K 31/137

(52) U.S. Cl. CPC C07C 217/60; A61K 31/137

(57) ABSTRACT

The present disclosure provides methods for the preparation of mescaline, including salts thereof through amination of 2-(3,4,5-trimethoxyphenyl)acetic acid with ammonia in a polar aprotic solvent, followed by reduction of the 3,4,5-trimethoxypheny-lacetamide with a mixture of LiAIH4 and AICI3 and a coupling agent carbonyldiimidazole. Also included are compositions comprising mescaline and synthetic intermediates.

### CROSS-REFERENCE

Priority is claimed under PCT Art. 8(1) and Rule 4.10 to U.S. Appl. No. 63/314,129, filed Feb. 25, 2022, and U.S. Appl. No. 63/345,602, filed May 25, 2022, both of which are incorporated by reference for all purposes as if fully set forth herein.

### TECHNICAL FIELD

Provided is a novel process for the preparation of mescaline, including salts thereof.

### BACKGROUND OF THE INVENTION

Naturally-derived psychedelic compounds from plants, fungi or other organisms are known to be effective at treating or alleviating various psychological and physiological conditions. For example, mescaline (3,4,5-trimethoxyphenethylamine) or extracts of Lophophora williamsii, Trichocereus (Echinopsis) pachanoi, Echinopsis peruviana, Trichocereus bridgesii, Pereskia aculeata, Acacia berlandieri, and Pelecyphora aselliformis have been shown to be effective in treating various psychological disorders in recent years. However, the shelf life of these products in their natural form is variable and creates a potential for inconsistency in the dosing of the active compound in the product. Additionally, the natural plant or plant extractions can have varying concentrations of an active compound leading to variability in dosing and potency. Due to this, several approaches have been developed to produce synthetically derived mescaline. However, these approaches often result in low yields of mescaline or are otherwise not suitable for large scale production (e.g., because of safety or processing issues). Thus, there is an ongoing need to develop methods for the large scale production of mescaline, including salts thereof, to enable the treatment of a wide variety of diseases and conditions.

### SUMMARY OF THE INVENTION

The following presents a simplified summary of some embodiments of the invention in order to provide a basic understanding of the invention. This summary is not an extensive overview of the invention. It is not intended to identify key or critical elements of the invention or to delineate the scope of the invention. Its sole purpose is to present some embodiments of the invention in a simplified form as a prelude to the more detailed description that is presented later.

In a first aspect, provided is a method of preparing a compound of Formula (III),

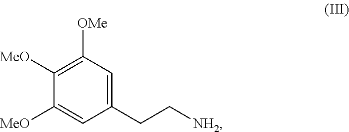 [SMILES: COc1cc(CCN)cc(OC)c1OC]

or a salt thereof, comprising: (i) subjecting a compound of Formula (I):

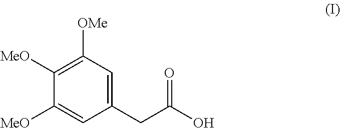 [SMILES: COc1cc(CC(=O)O)cc(OC)c1OC]

to amination conditions to form a compound of Formula (II):

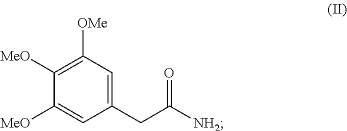 [SMILES: COc1cc(CC(N)=O)cc(OC)c1OC]

and (ii) subjecting the compound of Formula (II) to reducing conditions to produce the compound of Formula (III), or a salt thereof.

In some embodiments, step (i) is conducted in a polar aprotic solvent. In some embodiments, the polar aprotic solvent selected from the group consisting of acetone, acetonitrile, dichloromethane, chloroform, dimethylformamide, dimethylsulfoxide, ethyl acetate, tetrahydrofuran, diethyl ether, dioxane, furan, dimethoxyethane, methyl tert-butyl ether, and chlorobenzene. In some embodiments, the polar aprotic solvent is tetrahydrofuran.

In some embodiments (equivalently and as shorthand, “in embodiments”), the amination conditions comprise an amination reagent. In embodiments, the amination reagent is ammonia.

In some embodiments, the amination conditions further comprise a coupling agent. In some embodiments, the coupling agent is selected from the group consisting of dicyclohexylcarbodiimide (DCC), diisopropylcarbodiimide (DIC), 1-ethyl-3-(3-dimethylamino-propyl)carbodiimide (EDC), benzotriazol-1-yloxytris(dimethylamino)phosphonium hexafluorophosphate (BOP), 1-[bis(dimethylamino)methylene]-1H-1,2,3-triazolo[4,5-b]pyridinium 3-oxide hexafluorophosphate (HATU), carbonyldiimidazole (CDI), and carbonylditriazole. In some embodiments, the coupling agent is carbonyldiimidazole (CDI).

In some embodiments, the molar ratio of the compound of Formula (I) to the coupling agent is between about 1:1 and 1:5. In some embodiments, the molar ratio of the compound of Formula (I) to the coupling agent is about 1:1.2. In some embodiments, the coupling agent is added to the reaction mixture before the amination reagent. In some embodiments, the reaction temperature is between about 15° C. and 50° C. during step (i).

In some embodiments, step (ii) is conducted in a polar aprotic solvent. In some embodiments, the polar aprotic solvent selected from the group consisting of acetone, acetonitrile, dichloromethane, chloroform, dimethylformamide, dimethylsulfoxide, ethyl acetate, tetrahydrofuran, diethyl ether, dioxane, furan, dimethoxyethane, methyl tert-butyl ether, and chlorobenzene. In some embodiments, the polar aprotic solvent is tetrahydrofuran.

In some embodiments, the reducing conditions comprise a reducing agent. In some embodiments, the reducing agent is hydrogen or a hydride reagent. In some embodiments, the reducing agent is hydrogen, LiAlH₄, NaBH₄, or di-isobutyl aluminum hydride (DIBAL-H). In some embodiments, the reducing agent is LiAlH₄. In some embodiments, the reducing conditions further comprise a metal halide. In some embodiments, the metal halide is AlCl₃.

In some embodiments, the reducing conditions comprise LiAlH₄ and AlCl₃ in a molar ratio of between about 4:1 and 1:4. In some embodiments, the reducing conditions comprise LiAlH₄ and AlCl₃ in a molar ratio of about 3:1. In some embodiments, the reaction temperature is between about 35° C. and 100° C. during step (ii).

In some embodiments, the method comprises reacting the compound of Formula (III) with an acid to produce a compound of Formula (IV):

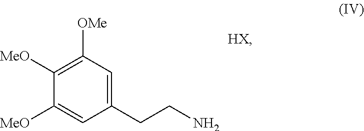

wherein X is the conjugate base of the acid.

In some embodiments, the acid is an inorganic acid. In some embodiments, the inorganic acid is selected from the group consisting of HCl, HBr, HI, HF, HNO₃, H₃PO₄, H₂SO₄, H₃BO₃, and HClO₄. In some embodiments, the inorganic acid is HCl, HBr, HI, or H₂SO₄. In some embodiments, the inorganic acid is HCl. In some embodiments, the ratio of the compound of Formula (III) to the inorganic acid is about 1:1 to about 1:100.

In some embodiments, reacting the compound of Formula (III) with an acid is conducted in a solvent selected from the group consisting of acetone, methanol, ethanol, propanol, isopropanol, n-butanol, isobutanol, tetrahydrofuran, isopropyl acetate, acetonitrile, and methyl ethyl ketone. In some embodiments, the solvent is isopropanol. In some embodiments, the method further comprises isolating the compound of Formula (IV) by filtration. In some embodiments, the method is performed under Good Manufacturing Practices (GMP) conditions.

In another aspect, provided is a composition comprising a compound of Formula (II),

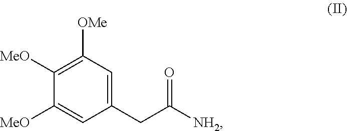 [SMILES: COc1cc(CC(N)=O)cc(OC)c1OC]

and any of: (a) between about 0.1 wt % and 5 wt % imidazole; (b) between about 0.1 wt % to 1.0 wt % dichloromethane; and (c) between about 0.1 wt % and 3.0 wt % 3,4,5-trimethoxyphenyl acetic acid.

In another aspect, provided is a composition comprising a compound of Formula (III),

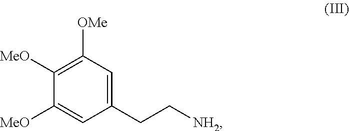 [SMILES: COc1cc(CCN)cc(OC)c1OC]

and any of: (a) between about 0.1 wt % and 8.0 wt % 2-(3,4,5-trimethoxyphenyl)acetamide; (b) between about 0.1 wt % and 5 wt % dimeric amine; (c) between about 0.1 wt % and 5 wt % n-butanol; and (d) between about 0.1 wt % and 6% toluene.

A composition comprising a compound of Formula (IV-a),

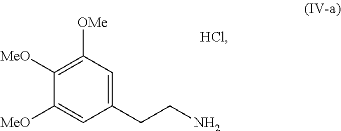 [SMILES: COc1cc(CCN)cc(OC)c1OC ; "HCl,"]

and any of: (a) between about 0.1 wt % and 8.0 wt % 2-(3,4,5-trimethoxyphenyl)acetamide; (b) between about 0.01 wt % and 5 wt % 4-(2-aminoethyl)-2,6-dimethoxyphenol; (c) between about 0.01 wt % and 5 wt % 5-(2-aminoethyl)-2,3-dimethoxyphenol; (d) between about 1 ppm and 50 ppm isopropanol; (e) between about 1 ppm and 50 ppm methanol; (f) between about 500 ppm and 1000 ppm ethanol; (g) between about 0.01 wt % and 0.25 wt % water; (h) between about 10 wt % and 20 wt % chloride; (i) between about 0.1 ppm and 10 ppm Li; (j) between about 0.1 ppm and 200 ppm Al; (k) between about 0.1 wt % and 8.0 wt % 2-(3,4,5-trimethoxyphenyl)acetamide; (l) between about 0.01 wt % and 5 wt % 4-(2-aminoethyl)-2,6-dimethoxyphenol; and (m) between about 0.01 wt % and 5 wt % 5-(2-aminoethyl)-2,3-dimethoxyphenol.

Also provided are pharmaceutical compositions comprising the composition of any of the preceding embodiments; and a pharmaceutically acceptable carrier, diluent, or excipient.

In a further aspect, provided is a method of treating a substance abuse disorder, comprising administering to a subject in need thereof the composition or the pharmaceutical composition of any of the preceding embodiments.

The foregoing has outlined broadly and in summary certain pertinent features of the disclosure so that the detailed description of the invention that follows may be better understood, and so that the present contribution to the art can be more fully appreciated. Hence, this summary is to be considered as a brief and general synopsis of only some of the objects and embodiments disclosed herein, is provided solely for the benefit and convenience of the reader, and is not intended to limit in any manner the scope, or range of equivalents, to which the claims are lawfully entitled. Additional features of the invention are described hereinafter. It should be appreciated by those in the art that all disclosed specific compositions and methods are only exemplary, and may be readily utilized as a basis for modifying or designing other compositions and methods for carrying out the same purposes. Such equivalent compositions and methods will be appreciated to be also within the scope and spirit of the invention as set forth in the claims. It also will be appreciated that headings within this document are being utilized only to expedite its review by a reader. They should not be construed as limiting the invention in any manner.

### INCORPORATION BY REFERENCE

Each patent, publication, and non-patent literature cited in the application or listed in the section titled “References” below, is hereby incorporated by reference in its entirety, as if each was incorporated by reference individually, and as if each is fully set forth herein. However, where such reference is made, and whether to patents, publications, non-patent literature, or other sources of information, it is for the general purpose of providing context for discussing features of the invention. Accordingly, unless specifically stated otherwise, the reference is not to be construed as an admission that the document or underlying information, in any jurisdiction, is prior art or forms part of the common general knowledge in the art.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms herein have the meaning as commonly understood by one having ordinary skill in the art to which this invention belongs, who as a shorthand may be referred to simply as “one of skill.” Further definitions that may assist the reader in understanding the disclosed embodiments are as follows; however, it will be appreciated that such definitions are not intended to limit the scope of the invention, which shall be properly interpreted and understood by reference to the full specification (as well as any plain meaning known to one of skill in the relevant art) in view of the language used in the appended claims. The terminology used herein is for the purpose of describing particular embodiments only and is not intended to be limiting.

Generally, the nomenclature used and procedures performed herein are those known in fields relating to one or more aspects of the invention, such as biology, pharmacology, neuroscience, organic chemistry, synthetic chemistry, and/or medicinal chemistry, and are those that will be well known and commonly employed in such fields. Standard techniques and procedures will be those generally performed according to conventional methods in the art.

As used in the specification and claims, the singular form “a”, “an” and “the” includes plural references unless the context clearly dictates otherwise.

Compounds of the present disclosure also include crystalline and amorphous forms of those compounds, pharmaceutically acceptable salts, and active metabolites of these compounds having the same type of activity, including, for example, polymorphs, pseudo-polymorphs, solvates, hydrates, unsolvated polymorphs (including anhydrates), conformational polymorphs, and amorphous forms of the compounds, as well as mixtures thereof. It should be understood that any reference to a disclosed compound or a pharmaceutically acceptable salt, prodrug, hydrate, or solvate thereof, will include all amorphous and polymorphic forms. In the case of solid compositions, in particular, it is understood that the compounds used in the disclosed compositions and methods may exist in different forms. For example, the compounds may exist in stable and metastable crystalline forms, isotropic and amorphous forms, milled forms and nano-particulate forms, all of which are intended to be within the scope of the invention. In addition, disclosed compounds may include crystalline forms, known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The compounds described herein may exhibit their natural isotopic abundance, or one or more of the atoms may be artificially enriched in a particular isotope having the same atomic number, but an atomic mass or mass number different from the atomic mass or mass number predominantly found in nature. Examples of isotopes that can be incorporated into compounds described herein include isotopes of hydrogen, carbon, nitrogen, oxygen, and chlorine such as ²H ³H, ¹¹C, ¹³C, ¹⁴C, ¹⁵N, ¹⁷O, ¹⁸O, and ³⁶Cl respectively. In one non-limiting embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention can generally be prepared by carrying out the procedures disclosed in the schemes or in the examples and preparations described below by substituting a readily available isotopically labeled reagent for a non-isotopically labeled reagent. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopic substitutions, for example deuterium substitutions, can be partial or complete. Partial deuterium substitution means that at least one hydrogen is substituted with deuterium. In certain embodiments, the isotope is at least 60, 70, 80, 90, 95, or 99% or more enriched in an isotope at any location of interest. In one non-limiting embodiment, deuterium is 90, 95, or 99% enriched at a desired location. Isotopically-enriched compounds may be prepared by conventional techniques well known to those skilled in the art.

The term “in vivo” refers to an event that takes place in a subject's body.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

Herein, “isopropanol,” “isopropyl alcohol,” and “propan-2-ol” are used interchangeably.

As described herein the products and intermediates synthesized can be analyzed using a variety of experimental techniques. For example, and not limited to, the method of elicitation can be Karl-Fischer titration (KF), ¹H-NMR, ¹³C-NMR, Mass Spectroscopy (MS), liquid chromatography (LC), Elemental Analysis, Inductively Coupled Plasma Atomic Emission Spectroscopy (ICP-OES), X-ray powder diffraction (XPRD), Differential Scanning Calorimetry (DSC), Gas chromatography (GC), or Thermogravimetric Analysis (TGA).

A comprehensive list of the abbreviations utilized by organic chemists of ordinary skill in the art appears in the first issue of each volume of the Journal of Organic Chemistry; this list is typically presented in a table entitled Standard List of Abbreviations; the current list as of the date of this filing is hereby incorporated by reference as if fully set forth herein.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier, diluent or excipient” includes without limitation any adjuvant, carrier, excipient, glidant, sweetening agent, diluent, preservative, dye, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Herein, “about” includes the recited number ±10%. Thus, “about 10” means 9 to 11.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “at most”, “less than,” or “less than or equal to” precedes the first numerical value in a series of two or more numerical values, the term “no more than,” “at most”, “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “decreased” or “decrease,” as used herein, generally mean a decrease by a statistically significant amount. In some embodiments, “decreased” or “decrease” means a reduction by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease from 10% to 100% as compared to a reference level. In the context of a marker or symptom, by these terms is meant a statistically significant decrease in such level. The decrease can be, for example, at least 10%, at least 20%, at least 30%, at least 40% or more.

Still additional definitions and abbreviations are provided elsewhere herein.

The section headings used herein are for organizational purposes only and are not to be construed as limiting the subject matter described. Any aspect or embodiment described herein can be combined with any other aspect or embodiment as disclosed herein.

### Methods of Producing Mescaline and Salts Thereof

Several methods of synthesizing mescaline have been published (see, e.g. Banholzer, et al. Helvetica Chimica Acta. 1952, 35(5), 1577-1581; Cassels, et al. ACS Chem. Neurosci. 2018, 9, 2448-2458). However, many of these approaches result in low yields of mescaline or are otherwise not suitable for large scale production (e.g., because of safety or processing issues). There is an ongoing need for safe, effective, and economical methods for synthesizing mescaline. Provided herein is a novel process for the preparation of mescaline, including salts thereof, to meet these needs and others, and having such advantages and improvements as will become readily apparent through the disclosure below

In certain aspects the present disclosure provides methods of synthesizing a compound of Formula (IV).

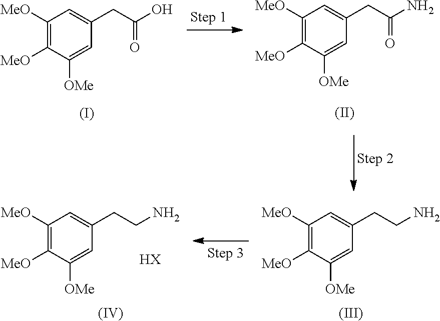

In some embodiments, the present disclosure provides methods of producing a compound of Formula (II). In some embodiments, the present disclosure provides methods of producing a compound of Formula (III). In some embodiments, the present disclosure provides methods of producing a compound of Formula (IV). In some embodiments, the method provided herein, is performed under Good Manufacturing Practices (GMP) conditions. In additional embodiments, the method is performed in a GMP facility.

In some embodiments, the present disclosure provides a compound of Formula (II). In some embodiments, the compound of Formula (II) comprises additional characteristics. In some embodiments, the compound of Formula (II) is produced using step 1. In some embodiments, the present disclosure provides a compound of Formula (III). In some embodiments, the compound of Formula (III) comprises additional characteristics. In some embodiments, the compound of Formula (III) is produced using step 2. In some embodiments, the present disclosure provides a compound of Formula (IV). In some embodiments, the compound of Formula (IV) comprises additional characteristics. In some embodiments, the compound of Formula (IV) is produced using step 3.

In some embodiments, the compound or salt of Formula (IV) may be formulated into a pharmaceutical composition. In some embodiments, the pharmaceutical composition is in a tablet or a capsule. In some embodiments, the pharmaceutical composition is a tablet. In some embodiments, the pharmaceutical composition is a capsule. In some embodiments, the pharmaceutical composition is in an oral dosage form.

In some embodiments, 80% or more of the pharmaceutical composition comprises the compound or salt of Formula (IV). In some embodiments, 90% or more of the pharmaceutical composition comprises the compound or salt of Formula (IV). In some embodiments, 95% or more of the pharmaceutical composition comprises the compound or salt of Formula (IV). In some embodiments, 80% or more, 85% or more, 90% or more, 95% or more, or 99% or more of the compound or salt of Formula (IV). In some embodiments, at least 80%, at least 85%, at least 90%, at least 95%, or at least 99% of the pharmaceutical composition comprises the compound or salt of Formula (IV).

The procedures described herein are useful for the production of synthetic mescaline and salts thereof. The methods described herein are advantageous over direct extraction from mescaline containing plants such as: Lophophora williamsii, Trichocereus (Echinopsis) pachanoi, Echinopsis peruviana, Trichocereus bridgesii, Pereskia aculeata, Acacia berlandieri, or Pelecyphora aselliformis. The compositions described herein are free of plant cells, plant tissue, and/or naturally occurring alkaloids. For example, the compositions disclosed herein, lack naturally occurring alkaloids associated with the extraction of mescaline from plants. In some embodiments, the naturally occurring alkaloid is selected from N-methylmescaline, N-acetylmescaline, hordenine, pellotine, anhalonine, lobivine, tyramine, N-methyltyramine, anhalidine, anhalonidine, O-methyl-anhalonidine, lophophine, lophophorine, anhalinine, anhalamine, homopiperonylamine, and a combination thereof. In some embodiments, the naturally occurring alkaloid is represented by:

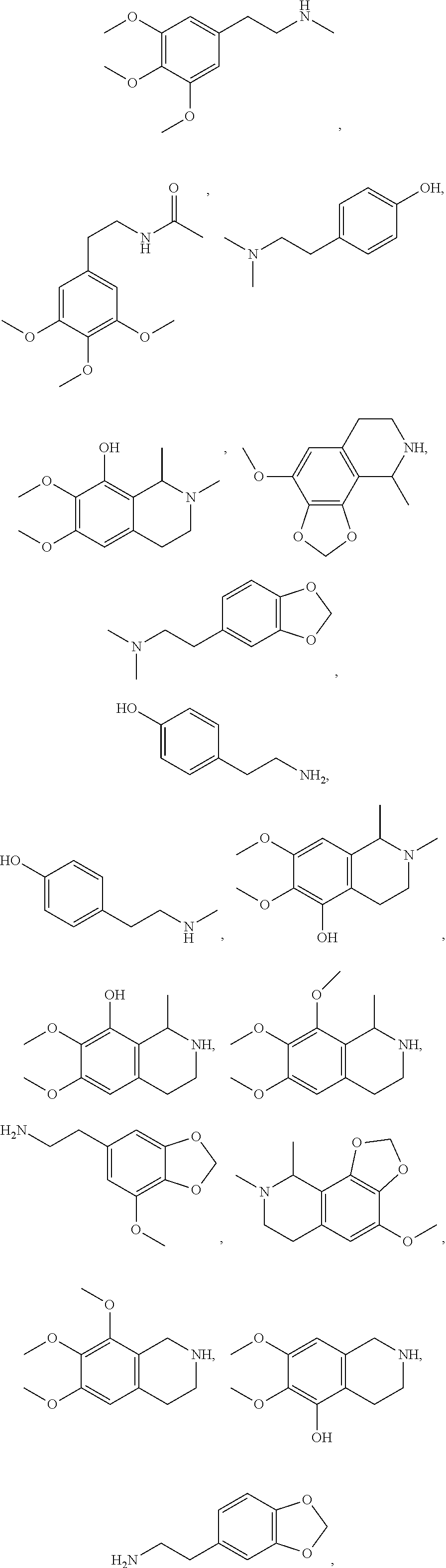

or combinations thereof.

### Step 1

The present disclosure provides a method for synthesizing a compound for Formula (II) comprising, contacting a compound represented by Formula (I)

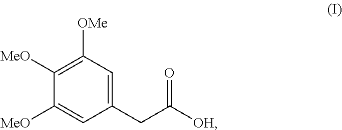 [SMILES: COc1cc(CC(=O)O)cc(OC)c1OC]

with a coupling agent and under amination conditions, wherein the by-product of the contacting is CO₂, to produce a compound of Formula (II),

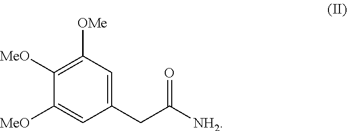 [SMILES: COc1cc(CC(N)=O)cc(OC)c1OC]

In some embodiments, the method comprises subjecting a compound of Formula (I):

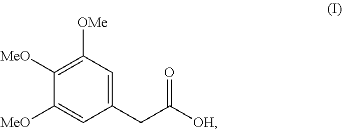 [SMILES: COc1cc(CC(=O)O)cc(OC)c1OC]

to amination conditions to form a compound of Formula (II):

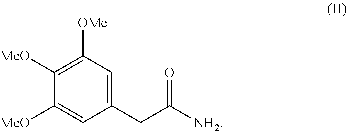 [SMILES: COc1cc(CC(N)=O)cc(OC)c1OC]

In some embodiments, the amination conditions comprise a solvent. In some embodiments, the solvent is selected from a polar aprotic solvent and a polar protic solvent. In some embodiments, the amination conditions comprise a polar protic solvent.

In some embodiments, the solvent is a polar aprotic solvent selected from acetone, acetonitrile, dichloromethane, chloroform, dimethylformamide, dimethylsulfoxide, ethyl acetate, tetrahydrofuran, diethyl ether, dioxane, furan, dimethoxyethane, methyl tert-butyl ether, and chlorobenzene. In some embodiments, the solvent is a polar aprotic solvent is selected from acetone, acetonitrile, dichloromethane, chloroform, dimethylformamide, dimethylsulfoxide, ethyl acetate, dioxane, pyran, furan, methyl tert-butyl ether, and chlorobenzene. In some embodiments, the solvent is a polar aprotic solvent selected from acetone, acetonitrile, dichloromethane, chloroform, dimethylformamide, and dimethylsulfoxide. In some embodiments, the solvent is a polar aprotic solvent selected from ethyl acetate, dioxane, pyran, furan, methyl tert-butyl ether, and chlorobenzene.

In some embodiments, the solvent is a polar aprotic solvent is selected from diethyl ether, tetrahydrofuran, and dimethoxyethane. In some embodiments, the polar aprotic solvent is diethyl ether. In some embodiments, the polar aprotic solvent is dimethoxyethane. In some embodiments, the polar aprotic solvent is tetrahydrofuran.

In some embodiments, the amination conditions comprise an amination reagent. An amination reagent is a reagent which can convert a suitable precursor to an amine-containing product (such as a primary amine, secondary amine, tertiary amine, a primary amide, a secondary amide, or a tertiary amide). In some embodiments, the amination reagent is a reagent that can convert a carboxylic acid precursor to an amide product, such as a primary amide. In some embodiments, the amination reagent is a reagent that can convert a carboxylic acid to an ammonium carboxylate salt, which subsequently dehydrates to produce a primary amide (i.e., RCOO⁻ NH₄⁺→RCONH₂+H₂O).

In some embodiments, the amination reagent is an ammonium salt, such as ammonium carbonate. In other, preferred embodiments, the amination reagent is ammonia. In some embodiments, the amination reagent is gaseous ammonia or ammonia in a solution. In some embodiments, the amination reagent is gaseous ammonia which is sparged into a reaction. In some embodiments, the gaseous ammonia is added to the solvent in excess.

In some embodiments, the amination conditions comprise sparging ammonia in the solvent. In some embodiments, the sparging ammonia in the solvent comprises a sparging time. In some embodiments, the sparging ammonia comprises a sparge time of about 5 minutes to about 60 minutes. In some embodiments, the sparging ammonia comprises a sparge time of about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, or about 75 minutes. In some embodiments, the sparging ammonia comprises a sparge time of at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, or at least 75 minutes. In some embodiments, the sparging ammonia comprises a sparge time of at most 5 minutes, at most 10 minutes, at most 15 minutes, at most 20 minutes, at most 25 minutes, at most 30 minutes, at most 35 minutes, at most 40 minutes, at most 45 minutes, at most 50 minutes, at most 55 minutes, at most 60 minutes, at most 65 minutes, at most 70 minutes, or at most 75 minutes.

In some embodiments, the sparging ammonia in the solvent comprises a sparging flow rate. In some embodiments, the sparging ammonia in the solvent comprises a sparging flow rate is between about 0.1 L/min to 2 L/min. In some embodiments, the sparging flow rate is about 0.1 L/min, about 0.2 L/min, about 0.3 L/min, about 0.4 L/min, about 0.5 L/min, about 0.6 L/min, about 0.7 L/min, about 0.8 L/min, about 0.9 L/min, about 1 L/min, about 1.1 L/min, about 1.2 L/min, about 1.3 L/min, about 1.4 L/min, about 1.5 L/min, about 1.6 L/min, about 1.7 L/min, about 1.8 L/min, about 1.9 L/min, or about 2 L/min. In some embodiments, the sparging flow rate is at least 0.1 L/min, at least 0.2 L/min, at least 0.3 L/min, at least 0.4 L/min, at least 0.5 L/min, at least 0.6 L/min, at least 0.7 L/min, at least 0.8 L/min, at least 0.9 L/min, at least 1 L/min, at least 1.1 L/min, at least 1.2 L/min, at least 1.3 L/min, at least 1.4 L/min, at least 1.5 L/min, at least 1.6 L/min, at least 1.7 L/min, at least 1.8 L/min, at least 1.9 L/min, or at least 2 L/min. In some embodiments, the sparging flow rate is at most 0.1 L/min, at most 0.2 L/min, at most 0.3 L/min, at most 0.4 L/min, at most 0.5 L/min, at most 0.6 L/min, at most 0.7 L/min, at most 0.8 L/min, at most 0.9 L/min, at most 1 L/min, at most 1.1 L/min, at most 1.2 L/min, at most 1.3 L/min, at most 1.4 L/min, at most 1.5 L/min, at most 1.6 L/min, at most 1.7 L/min, at most 1.8 L/min, at most 1.9 L/min, or at most 2 L/min.

In some embodiments, the method comprises a coupling agent. A coupling agent is a reagent that can promote the conversion of a carboxylic acid into another product, such as an amide or an ester. In embodiments, the coupling agent promotes the conversion of a carboxylic acid into an amide. In embodiments, the coupling agent promotes the conversion of a carboxylic acid into an amide by reacting with the carboxylic acid to form an activated acyl intermediate. Some non-limiting examples of activated acyl intermediates include acyl halides (such as an acyl chloride or acyl bromide), O-acylisoureas (such as those formed by the reaction of a carboxylic acid with a carbodiimide coupling reagent), N-hydroxysuccinimide esters, and acyl imidazoles (such as those formed by the reaction of a carboxylic acid with carbonyldiimidazole).

In some embodiments, the coupling agent is selected from Dicyclohexylcarbodiimide (DCC), Diisopropylcarbodiimide (DIC), 1-Ethyl-3-(3-dimethylaminopropyl)carbodiimide (EDC), benzotriazol-1-yloxytris(dimethylamino)phosphonium hexafluorophosphate (BOP), 1-[Bis(dimethylamino)methylene]-1H-1,2,3-triazolo[4,5-b]pyridinium 3-oxide hexafluorophosphate (HATU), carbonyldiimidazole (CDI), and carbonylditriazole. In embodiments, the coupling agent is carbonyldiimidazole or carbonylditriazole. In some embodiments, the coupling agent is carbonylditriazole. In some embodiments, the coupling agent is carbonyldiimidazole (CDI). In some preferred embodiments, CDI is used in tandem with ammonia in disclosed methods to convert the compound of Formula (I) to the compound of Formula (II).

In some embodiments, the amount of CO₂ produced in Step 1 is about 0.01 moles (mol) to about 10 moles CO₂. In some embodiments, the amount of CO₂ produced in Step 1 is about 0.01 mol, about 0.02 mol, about 0.04 mol, about 0.05 mol, about 0.1 mol, about 0.2 mol, about 0.3 mol, about 0.4 mol, about 0.5 mol, about 0.6 mol, about 0.7 mol, about 0.8 mol, about 0.9 mol, about 1 mol, about 1.5 mol, about 2 mol, about 2.5 mol, about 3 mol, about 3.5 mol, about 4 mol, about 4.5 mol, about 5 mol, about 5.5 mol, about 6 mol, about 6.5 mol, about 7 mol, about 7.5 mol, about 8 mol, about 8.5 mol, about 9 mol, about 9.5 mol, about 10 mol, about 15 mol, about 20 mol, or about 25 mol. In some embodiments, the amount of CO₂ produced in Step 1 is at least 0.01 mol, at least 0.02 mol, at least 0.04 mol, at least 0.05 mol, at least 0.1 mol, at least 0.2 mol, at least 0.3 mol, at least 0.4 mol, at least 0.5 mol, at least 0.6 mol, at least 0.7 mol, at least 0.8 mol, at least 0.9 mol, at least 1 mol, at least 1.5 mol, at least 2 mol, at least 2.5 mol, at least 3 mol, at least 3.5 mol, at least 4 mol, at least 4.5 mol, at least 5 mol, at least 5.5 mol, at least 6 mol, at least 6.5 mol, at least 7 mol, at least 7.5 mol, at least 8 mol, at least 8.5 mol, at least 9 mol, at least 9.5 mol, at least 10 mol, at least 15 mol, at least 20 mol, or at least 25 mol. In some embodiments, the amount of CO₂ produced in Step 1 is at most 0.01 mol, at most 0.02 mol, at most 0.04 mol, at most 0.05 mol, at most 0.1 mol, at most 0.2 mol, at most 0.3 mol, at most 0.4 mol, at most 0.5 mol, at most 0.6 mol, at most 0.7 mol, at most 0.8 mol, at most 0.9 mol, at most 1 mol, at most 1.5 mol, at most 2 mol, at most 2.5 mol, at most 3 mol, at most 3.5 mol, at most 4 mol, at most 4.5 mol, at most 5 mol, at most 5.5 mol, at most 6 mol, at most 6.5 mol, at most 7 mol, at most 7.5 mol, at most 8 mol, at most 8.5 mol, at most 9 mol, at most 9.5 mol, at most 10 mol, at most 15 mol, at most 20 mol, or at most 25 mol.

In some embodiments, the amination conditions comprise maintaining a reaction temperature of about 15° C. to about 50° C. In some embodiments, the amination conditions comprise maintaining a reaction temperature of about 10° C., about 15° C., about 20° C., about 25° C., about 30° C., about 35° C., about 40° C., about 45° C., or about 50° C. In some embodiments, the amination conditions comprise maintaining a reaction temperature of at least 10° C., at least 15° C., at least 20° C., at least 25° C., at least 30° C., at least 35° C., at least 40° C., at least 45° C., or at least 50° C. In some embodiments, the amination conditions comprise maintaining a reaction temperature of at most 10° C., at most 15° C., at most 20° C., at most 25° C., at most 30° C., at most 35° C., at most 40° C., at most 45° C., or at most 50° C.

In some embodiments, the molar ratio of the compound of Formula (I) to the coupling agent is about 1:1 to about 1:5. In some embodiments, the molar ratio of the compound of Formula (I) to the coupling agent is about 1:1, about 1:1.5, about 1:2, about 1:2.5, about 1:3, about 1:3.5, about 1:4, about 1:4.5, or about 1:5. In some embodiments, the molar ratio of the compound of Formula (I) to the coupling agent is at least 1:1, at least 1:1.5, at least 1:2, at least 1:2.5, at least 1:3, at least 1:3.5, at least 1:4, at least 1:4.5, or at least 1:5. In embodiments, the molar ratio of the compound of Formula (I) to the coupling agent is at most 1:1, at most 1:1.5, at most 1:2, at most 1:2.5, at most 1:3, at most 1:3.5, at most 1:4, at most 1:4.5, or at most 1:5.

In some embodiments, the molar ratio of the compound of Formula (I) to carbonyldiimidazole is about 1:1 to about 1:5. In some embodiments, the molar ratio of the compound of Formula (I) to carbonyldiimidazole is about 1:1, about 1:1.5, about 1:2, about 1:2.5, about 1:3, about 1:3.5, about 1:4, about 1:4.5, or about 1:5. In some embodiments, the molar ratio of the compound of Formula (I) to carbonyldiimidazole is at least 1:1, at least 1:1.5, at least 1:2, at least 1:2.5, at least 1:3, at least 1:3.5, at least 1:4, at least 1:4.5, or at least 1:5. In some embodiments, the molar ratio of the compound of Formula (I) to carbonyldiimidazole is at most 1:1, at most 1:1.5, at most 1:2, at most 1:2.5, at most 1:3, at most 1:3.5, at most 1:4, at most 1:4.5, or at most 1:5. In some embodiments, the molar ratio of the compound of Formula (I) to the coupling agent is between about 1:1 and 1:5. In some embodiments, the molar ratio of the compound of Formula (I) to the coupling agent is about 1:1.2. In preferred embodiments, wherein the coupling agent is carbonyldiimidazole, the molar ratio of the compound of Formula (I) to the carbonyldiimidazole is about 1:1.2.

In some embodiments, the amination conditions comprise a first reaction time from about 15 minutes to about 250 minutes. In some embodiments, the first reaction time from about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 165 minutes, about 170 minutes, about 175 minutes, about 180 minutes, about 185 minutes, about 190 minutes, about 195 minutes, about 200 minutes, about 205 minutes, about 210 minutes, about 215 minutes, about 220 minutes, about 225 minutes, about 230 minutes, about 235 minutes, about 240 minutes, about 245 minutes, about 250 minutes, about 255 minutes, about 260 minutes, about 265 minutes, or about 270 minutes. In some embodiments, the first reaction time from at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, at least 120 minutes, at least 125 minutes, at least 130 minutes, at least 135 minutes, at least 140 minutes, at least 145 minutes, at least 150 minutes, at least 155 minutes, at least 160 minutes, at least 165 minutes, at least 170 minutes, at least 175 minutes, at least 180 minutes, at least 185 minutes, at least 190 minutes, at least 195 minutes, at least 200 minutes, at least 205 minutes, at least 210 minutes, at least 215 minutes, at least 220 minutes, at least 225 minutes, at least 230 minutes, at least 235 minutes, at least 240 minutes, at least 245 minutes, at least 250 minutes, at least 255 minutes, at least 260 minutes, at least 265 minutes, or at least 270 minutes. In some embodiments, the first reaction time from at most 5 minutes, at most 10 minutes, at most 15 minutes, at most 20 minutes, at most 25 minutes, at most 30 minutes, at most 35 minutes, at most 40 minutes, at most 45 minutes, at most 50 minutes, at most 55 minutes, at most 60 minutes, at most 65 minutes, at most 70 minutes, at most 75 minutes, at most 80 minutes, at most 85 minutes, at most 90 minutes, at most 95 minutes, at most 100 minutes, at most 105 minutes, at most 110 minutes, at most 115 minutes, at most 120 minutes, at most 125 minutes, at most 130 minutes, at most 135 minutes, at most 140 minutes, at most 145 minutes, at most 150 minutes, at most 155 minutes, at most 160 minutes, at most 165 minutes, at most 170 minutes, at most 175 minutes, at most 180 minutes, at most 185 minutes, at most 190 minutes, at most 195 minutes, at most 200 minutes, at most 205 minutes, at most 210 minutes, at most 215 minutes, at most 220 minutes, at most 225 minutes, at most 230 minutes, at most 235 minutes, at most 240 minutes, at most 245 minutes, at most 250 minutes, at most 255 minutes, at most 260 minutes, at most 265 minutes, or at most 270 minutes.

In some embodiments, the initial concentration of the compound of Formula (I) in the solvent is about 0.05 Moles/Liters (mol/L) to about 2 Moles/Liters prior to the addition of the coupling agent or the amination conditions. In some embodiments, the initial concentration of the compound of Formula (I) in the solvent is about 0.05 mol/L, about 0.10 mol/L, about 0.15 mol/L, about 0.20 mol/L, about 0.25 mol/L, about 0.30 mol/L, about 0.35 mol/L, about 0.40 mol/L, about 0.45 mol/L, about 0.50 mol/L, about 0.55 mol/L, about 0.60 mol/L, about 0.65 mol/L, about 0.70 mol/L, about 0.75 mol/L, about 0.80 mol/L, about 0.85 mol/L, about 0.90 mol/L, about 0.95 mol/L, about 1.00 mol/L, about 1.25 mol/L, about 1.50 mol/L, about 1.75 mol/L, or about 2.00 mol/L. In some embodiments, the initial concentration of the compound of Formula (I) in the solvent is at least 0.05 mol/L, at least 0.10 mol/L, at least 0.15 mol/L, at least 0.20 mol/L, at least 0.25 mol/L, at least 0.30 mol/L, at least 0.35 mol/L, at least 0.40 mol/L, at least 0.45 mol/L, at least 0.50 mol/L, at least 0.55 mol/L, at least 0.60 mol/L, at least 0.65 mol/L, at least 0.70 mol/L, at least 0.75 mol/L, at least 0.80 mol/L, at least 0.85 mol/L, at least 0.90 mol/L, at least 0.95 mol/L, at least 1.00 mol/L, at least 1.25 mol/L, at least 1.50 mol/L, at least 1.75 mol/L, or at least 2.00 mol/L. In some embodiments, the initial concentration of the compound of Formula (I) in the solvent is at most 0.05 mol/L, at most 0.10 mol/L, at most 0.15 mol/L, at most 0.20 mol/L, at most 0.25 mol/L, at most 0.30 mol/L, at most 0.35 mol/L, at most 0.40 mol/L, at most 0.45 mol/L, at most 0.50 mol/L, at most 0.55 mol/L, at most 0.60 mol/L, at most 0.65 mol/L, at most 0.70 mol/L, at most 0.75 mol/L, at most 0.80 mol/L, at most 0.85 mol/L, at most 0.90 mol/L, at most 0.95 mol/L, at most 1.00 mol/L, at most 1.25 mol/L, at most 1.50 mol/L, at most 1.75 mol/L, or at most 2.00 mol/L. In some embodiments, the initial concentration of the compound of Formula (I) in the solvent is between about 0.25 mol/L to about 1.00 mol/L. In some embodiments, the initial concentration of the compound of Formula (I) in the solvent is between about 0.25 mol/L to about 0.75 mol/L. In some embodiments, the initial concentration of the compound of Formula (I) in the solvent is between about 0.25 mol/L to about 0.50 mol/L. In some embodiments, the initial concentration of the compound of Formula (I) in the solvent is between about 0.30 mol/L to about 0.50 mol/L. In some embodiments, the initial concentration of the compound of Formula (I) in the solvent is between about 0.35 mol/L to about 0.50 mol/L. In some embodiments, the initial concentration of the compound of Formula (I) in the solvent is between about 0.40 mol/L to about 0.50 mol/L.

In some embodiments, the initial concentration of the compound of Formula (I) in tetrahydrofuran is about 0.05 Moles/Liters (mol/L) to about 2 Moles/Liters prior to the addition of the coupling agent or the amination conditions. In some embodiments, the initial concentration of the compound of Formula (I) in tetrahydrofuran is about 0.05 mol/L, about 0.10 mol/L, about 0.15 mol/L, about 0.20 mol/L, about 0.25 mol/L, about 0.30 mol/L, about 0.35 mol/L, about 0.40 mol/L, about 0.45 mol/L, about 0.50 mol/L, about 0.55 mol/L, about 0.60 mol/L, about 0.65 mol/L, about 0.70 mol/L, about 0.75 mol/L, about 0.80 mol/L, about 0.85 mol/L, about 0.90 mol/L, about 0.95 mol/L, about 1.00 mol/L, about 1.25 mol/L, about 1.50 mol/L, about 1.75 mol/L, or about 2.00 mol/L. In some embodiments, the initial concentration of the compound of Formula (I) in tetrahydrofuran is at least 0.05 mol/L, at least 0.10 mol/L, at least 0.15 mol/L, at least 0.20 mol/L, at least 0.25 mol/L, at least 0.30 mol/L, at least 0.35 mol/L, at least 0.40 mol/L, at least 0.45 mol/L, at least 0.50 mol/L, at least 0.55 mol/L, at least 0.60 mol/L, at least 0.65 mol/L, at least 0.70 mol/L, at least 0.75 mol/L, at least 0.80 mol/L, at least 0.85 mol/L, at least 0.90 mol/L, at least 0.95 mol/L, at least 1.00 mol/L, at least 1.25 mol/L, at least 1.50 mol/L, at least 1.75 mol/L, or at least 2.00 mol/L. In some embodiments, the initial concentration of the compound of Formula (I) in tetrahydrofuran is at most 0.05 mol/L, at most 0.10 mol/L, at most 0.15 mol/L, at most 0.20 mol/L, at most 0.25 mol/L, at most 0.30 mol/L, at most 0.35 mol/L, at most 0.40 mol/L, at most 0.45 mol/L, at most 0.50 mol/L, at most 0.55 mol/L, at most 0.60 mol/L, at most 0.65 mol/L, at most 0.70 mol/L, at most 0.75 mol/L, at most 0.80 mol/L, at most 0.85 mol/L, at most 0.90 mol/L, at most 0.95 mol/L, at most 1.00 mol/L, at most 1.25 mol/L, at most 1.50 mol/L, at most 1.75 mol/L, or at most 2.00 mol/L. In some embodiments, the initial concentration of the compound of Formula (I) in tetrahydrofuran is between about 0.25 mol/L to about 1.00 mol/L. In some embodiments, the initial concentration of the compound of Formula (I) in the solvent is between about 0.25 mol/L to about 0.75 mol/L. In some embodiments, the initial concentration of the compound of Formula (I) in tetrahydrofuran is between about 0.25 mol/L to about 0.50 mol/L. In some embodiments, the initial concentration of the compound of Formula (I) in tetrahydrofuran is between about 0.30 mol/L to about 0.50 mol/L. In some embodiments, the initial concentration of the compound of Formula (I) in tetrahydrofuran is between about 0.35 mol/L to about 0.50 mol/L. In embodiments, the initial concentration of the compound of Formula (I) in tetrahydrofuran is between about 0.40 mol/L to about 0.50 mol/L.

In another aspect, the present disclosure provides a composition comprising a compound represented by Formula (II):

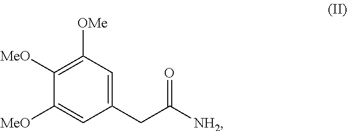 [SMILES: COc1cc(CC(N)=O)cc(OC)c1OC]

wherein the composition is characterized by one or more additional characteristics selected from: (i) between about 0.1 wt % to about 10 wt % imidazole content; (ii) between about 0.1 wt % to about 1.0 wt % dichloromethane content; and between about 0.1 wt % to about 3.0 wt % 3,4,5-Trimethoxyphenyl acetic acid. In some embodiments, the compositions comprising Formula (II) is characterized by at least two characteristics. In some embodiments, the compositions comprising Formula (II) is characterized by three characteristics.

In some embodiments, a composition comprising a compound represented by Formula (II):

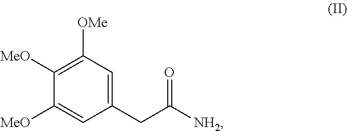 [SMILES: COc1cc(CC(N)=O)cc(OC)c1OC]

the composition is characterized by one or more additional characteristics selected from: (i) at least 0.1 wt % imidazole content; (ii) at most 1.0 wt % dichloromethane content; and at most 3.0 wt % 3,4,5-Trimethoxyphenyl acetic acid. In some embodiments, the compositions comprising Formula (II) is characterized by at least two characteristics. In some embodiments, the compositions comprising Formula (II) is characterized by three characteristics.

In some embodiments, a composition comprising a compound represented by Formula (II):

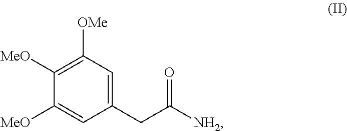 [SMILES: COc1cc(CC(N)=O)cc(OC)c1OC]

wherein the composition is characterized by one or more additional characteristics selected from: (i) between 0.1 wt % to 10 wt % imidazole content; (ii) between 0.1 wt % to 1.0 wt % dichloromethane content; and between 0.1 wt % to 3.0 wt % 3,4,5-Trimethoxyphenyl acetic acid. In some embodiments, the compositions comprising Formula (II) is characterized by at least two characteristics. In some embodiments, the compositions comprising Formula (II) is characterized by three characteristics.

### Step 2

In some aspects the present disclosure provides a method of synthesizing a compound of Formula (III). In some aspects the present disclosure provides a method of synthesizing a compound of Formula (III) comprising, subjecting the compound of Formula (II) to reducing conditions to produce a compound of Formula (III),

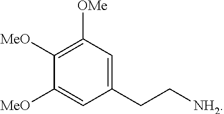 [SMILES: COc1cc(CCN)cc(OC)c1OC]

In some embodiments, the reducing conditions comprise a reducing agent. In some embodiments, the reducing agent is a hydrogen donor, hydrogen, a metal hydride, isopropyl alcohol, formic acid, or benzthiazoline. In some embodiments, the reducing agent is hydrogen or a hydride reagent. In some embodiments, the reducing agent is a hydrogen donor, hydrogen, or a metal hydride. In some embodiments, the reducing agent is selected from LiAlH₄, hydrogen, NaBH₄, di-isobutyl aluminum hydride (DIBAL-H), oxalic acid, and formic acid. the reducing agent is hydrogen, LiAlH₄, NaBH₄, or di-isobutyl aluminum hydride (DIBAL-H). In some embodiments, the reducing agent is selected from LiAlH₄, hydrogen, and NaBH₄. In some embodiments, the reducing agent is selected from LiAlH₄, hydrogen, NaBH₄, di-isobutyl aluminum hydride (DIBAL-H), oxalic acid, and formic acid. In some embodiments, the reducing agent is selected from LiAlH₄, hydrogen, and NaBH₄. In some embodiments, the reducing agent is selected from di-isobutyl aluminum hydride (DIBAL-H), oxalic acid, and formic acid. In some embodiments, the reducing agent is hydrogen. In some embodiments, the reducing agent is NaBH₄. In some embodiments, the reducing agent is LiAlH₄.

In some embodiments, the molar ratio of the compound of Formula (II) to LiAlH₄ is about 1:1 to about 1:5. In some embodiments, the molar ratio of the compound of Formula (II) to LiAlH₄ is about 1:1, about 1:1.5, about 1:2, about 1:2.5, about 1:3, about 1:3.5, about 1:4, about 1:4.5, or about 1:5. In some embodiments, the molar ratio of the compound of Formula (II) to LiAlH₄ is at least 1:1, at least 1:1.5, at least 1:2, at least 1:2.5, at least 1:3, at least 1:3.5, at least 1:4, at least 1:4.5, or at least 1:5. In some embodiments, the molar ratio of the compound of Formula (II) to LiAlH₄ is at most 1:1, at most 1:1.5, at most 1:2, at most 1:2.5, at most 1:3, at most 1:3.5, at most 1:4, at most 1:4.5, or at most 1:5.

In some embodiments, the reducing conditions further comprise a metal halide. In some embodiments, the metal halide is selected from AlCl₃, GaCl₃ InCl₃, FeCl₃, LaCl₃, CuCl₂, NiCl₂, AlBr₃, and AlI₃. In some embodiments, the metal halide is selected from AlCl₃, FeCl₃, AlBr₃, and AlI₃. In some embodiments, the reducing conditions further comprise AlCl₃.

In embodiments, the molar ratio of LiAlH₄ to AlCl₃ is from about 4:1 to about 1:4. In some embodiments, the molar ratio of LiAlH₄ to AlCl₃ is about 5:1, about 4.5:1, about 4:1, about 3.5:1, about 3:1, about 2.5:1, about 2:1, about 1.5:1, about 1:1, about 1:0.5, about 1:1.5, about 1:2, about 1:2.5, about 1:3, about 1:3.5, or about 1:4. In embodiments, the molar ratio of LiAlH₄ to AlCl₃ is at least 5:1, at least 4.5:1, at least 4:1, at least 3.5:1, at least 3:1, at least 2.5:1, at least 2:1, at least 1.5:1, at least 1:1, at least 1:0.5, at least 1:1.5, at least 1:2, at least 1:2.5, at least 1:3, at least 1:3.5, or at least 1:4. In some embodiments, the molar ratio of LiAlH₄ to AlCl₃ is at most 5:1, at most 4.5:1, at most 4:1, at most 3.5:1, at most 3:1, at most 2.5:1, at most 2:1, at most 1.5:1, at most 1:1, at most 1:0.5, at most 1:1.5, at most 1:2, at most 1:2.5, at most 1:3, at most 1:3.5, or at most 1:4. In preferred embodiments, the molar ratio of LiAlH₄ to AlCl₃ is about 3:1.

In some embodiments, the reducing conditions comprise a solvent. In some embodiments, the solvent is selected from a polar aprotic solvent and a polar protic solvent. In embodiments, the reducing conditions comprise a solvent selected from a polar protic solvent.

In some embodiments, the reducing conditions comprise a solvent selected from a polar aprotic solvent. In some embodiments, the solvent is a polar aprotic solvent selected from acetone, acetonitrile, dichloromethane, chloroform, dimethylformamide, dimethylsulfoxide, ethyl acetate, tetrahydrofuran, diethyl ether, dioxane, furan, dimethoxyethane, methyl tert-butyl ether, and chlorobenzene. In some embodiments, the solvent is a polar aprotic solvent is selected from acetone, acetonitrile, dichloromethane, chloroform, dimethylformamide, dimethylsulfoxide, ethyl acetate, dioxane, pyran, furan, methyl tert-butyl ether, and chlorobenzene. In some embodiments, the solvent is a polar aprotic solvent selected from acetone, acetonitrile, dichloromethane, chloroform, dimethylformamide, and dimethylsulfoxide. In some embodiments, the solvent is a polar aprotic solvent selected from ethyl acetate, dioxane, pyran, furan, methyl tert-butyl ether, and chlorobenzene.

In some embodiments, the solvent is a polar aprotic solvent is selected from diethyl ether, tetrahydrofuran, and dimethoxyethane. In some embodiments, the polar aprotic solvent is diethyl ether. In some embodiments, the polar aprotic solvent is dimethoxyethane. In embodiments, the polar aprotic solvent is tetrahydrofuran. In embodiments, the solvent is tetrahydrofuran.

In some embodiments, the reducing conditions comprise a second reaction temperature. In some embodiments, the second reaction temperature is from about 35° C. to about 100° C. In some embodiments, the second reaction temperature is about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., or about 130° C. In some embodiments, the second reaction temperature is at least 15° C., at least 20° C., at least 25° C., at least 30° C., at least 35° C., at least 40° C., at least 45° C., at least 50° C., at least 55° C., at least 60° C., at least 65° C., at least 70° C., at least 75° C., at least 80° C., at least 85° C., at least 90° C., at least 95° C., at least 100° C., at least 105° C., at least 110° C., at least 115° C., at least 120° C., at least 125° C., or at least 130° C. In some embodiments, the second reaction temperature is at most 15° C., at most 20° C., at most 25° C., at most 30° C., at most 35° C., at most 40° C., at most 45° C., at most 50° C., at most 55° C., at most 60° C., at most 65° C., at most 70° C., at most 75° C., at most 80° C., at most 85° C., at most 90° C., at most 95° C., at most 100° C., at most 105° C., at most 110° C., at most 115° C., at most 120° C., at most 125° C., or at most 130° C.

In embodiments, the reducing conditions comprise refluxing. In some embodiments, the refluxing lasts from about 1 hour to about 24 hours. In embodiments, the refluxing lasts about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In some embodiments, the refluxing lasts at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

In some embodiments, the refluxing lasts at most 1 hour, at most 2 hours, at most 3 hours, at most 4 hours, at most 5 hours, at most 6 hours, at most 7 hours, at most 8 hours, at most 9 hours, at most 10 hours, at most 11 hours, at most 12 hours, at most 13 hours, at most 14 hours, at most 15 hours, at most 16 hours, at most 17 hours, at most 18 hours, at most 19 hours, at most 20 hours, at most 21 hours, at most 22 hours, at most 23 hours, or at most 24 hours.

In another aspect, the present disclosure provides a composition comprising a compound represented by Formula (III):

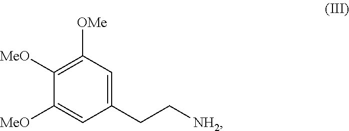 [SMILES: COc1cc(CCN)cc(OC)c1OC]

wherein the composition is characterized by one or more additional characteristics selected from: from: (i) between about 0.1 wt % to about 8.0 wt % 2-(3,4,5-trimethoxyphenyl)acetamide; (ii) between about 0.1 wt % to about 5 wt % dimeric amine; (iii) between about 0.1 wt % to about 5 wt % n-butanol; and (iv) between about 0.1 wt % to about 6% toluene. In some embodiments, the composition is characterized by two or more characteristics selected from i to iv. In some embodiments, the composition is characterized by at least two or more characteristics selected from i to iv. In some embodiments, the composition is characterized by at least three or more characteristics selected from i to iv. In some embodiments, the composition is characterized by at least four or more characteristics selected from i to iv. In embodiments, the composition is characterized by at least five or more characteristics selected from i to iv. In embodiments, the composition is characterized by at least six characteristics selected from i to iv. In embodiments, 2-(3,4,5-trimethoxyphenyl)acetamide is represented by

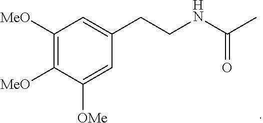 [SMILES: COc1cc(CCNC(C)=O)cc(OC)c1OC]

In embodiments, 2-(3,4,5-trimethoxyphenyl)acetamide is between about 0.1 wt % to about 7.0 wt %. In some embodiments, 2-(3,4,5-trimethoxyphenyl)acetamide is between about 0.1 wt % to about 6.0 wt %. In some embodiments, 2-(3,4,5-trimethoxyphenyl)acetamide is between about 0.1 wt % to about 5.0 wt %. In some embodiments, 2-(3,4,5-trimethoxyphenyl)acetamide is between 0.1 wt % to 7.0 wt %. In some embodiments, 2-(3,4,5-trimethoxyphenyl)acetamide is between 0.1 wt % to 6.0 wt %. In some embodiments, 2-(3,4,5-trimethoxyphenyl)acetamide is between 0.1 wt % to about 5.0 wt %.

In another aspect, the present disclosure provides a composition comprising a compound represented by Formula (III):

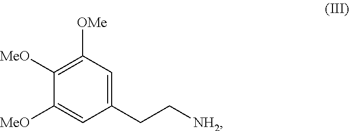 [SMILES: COc1cc(CCN)cc(OC)c1OC]

wherein the composition is characterized by one or more additional characteristics selected from: from: (i) between 0.1 wt % to 8.0 wt % 2-(3,4,5-trimethoxyphenyl)acetamide; (ii) between 0.1 wt % to 5 wt % dimeric amine; (iii) between 0.1 wt % to 5 wt % n-butanol; and (iv) between 0.1 wt % to 6% toluene. In some embodiments, the composition is characterized by two or more characteristics selected from i to iv. In some embodiments, the composition is characterized by at least two or more characteristics selected from i to iv. In some embodiments, the composition is characterized by at least three or more characteristics selected from i to iv. In some embodiments, the composition is characterized by at least four or more characteristics selected from i to iv. In some embodiments, the composition is characterized by at least five or more characteristics selected from i to iv. In some embodiments, the composition is characterized by at least six characteristics selected from i to iv.

In embodiments, 2-(3,4,5-trimethoxyphenyl)acetamide is represented by

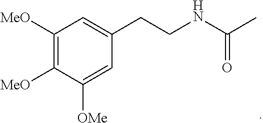 [SMILES: COc1cc(CCNC(C)=O)cc(OC)c1OC]

In some embodiments, 2-(3,4,5-trimethoxyphenyl)acetamide is between 0.1 wt % to 7.0 wt %. In some embodiments, 2-(3,4,5-trimethoxyphenyl)acetamide is between 0.1 wt % to 6.0 wt %. In some embodiments, 2-(3,4,5-trimethoxyphenyl)acetamide is between 0.1 wt % to about 5.0 wt %.

### Step 3

In some aspects the present disclosure provides a method of synthesizing a compound of Formula (IV). In some aspects the present disclosure provides a method of synthesizing a compound of Formula (IV) comprising, contacting the compound of Formula (III) with an acid under acidifying conditions to produce a compound of Formula (IV),

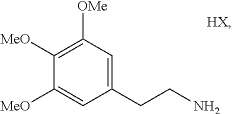

wherein X represents the conjugate base of the acid.

It will be understood that while, in various embodiments herein, a compound of Formula (IV) or any of its subformulae may be depicted as shown above, with a neutral primary amine moiety (—NH₂) and a neutral acid moiety (HX), the disclosure also encompasses the charged ammonium (—NH₃⁺) and conjugate base (X⁻) species in association with each other. The protonation state of the moieties of Formula (IV) can vary based on the identity of the acid as well as other factors known to one of skill in the art, and the equilibrium between neutral (i.e., uncharged) and charged species can be represented as follows:

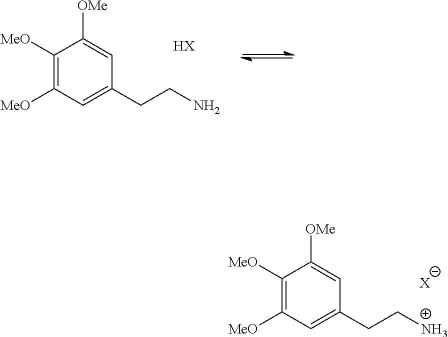

In some embodiments, the acid is an organic acid or an inorganic acid. In some embodiments, the acid is an organic acid. In some embodiments, the organic acid is selected from carboxylic acid, formic acid, acetic acid, benzoic acid, trifluoroacetic acid, phthalic acid, fumaric acid, oxalic acid, tartaric acid, a tartaric acid derivative, mandelic acid, maleic acid, citric acid, succinic acid, or malic acid, sulfonic acid, methanesulfonic acid, trifluoromethanesulfonic acid, benzenesulfonic acid, p-toluenesulfonic acid, and camphorsulfonic acid. In some embodiments, the acid is an inorganic acid. In some embodiments, the inorganic acid is selected from hydrofluoric acid, hydrochloric acid, hydrobromic acid, hydriodic acid, nitric acid, sulfuric acid, sulfurous acid, phosphoric acid, phosphorous acid, carbonic acid, and bicarbonic acid.

In some embodiments, the inorganic acid is selected from: HCl, HBr, HI, HF, HNO₃, H₃PO₄, H₂SO₄, H₃BO₃, and HClO₄. In some embodiments, the inorganic acid is selected from: HNO₃, H₃PO₄, H₂SO₄, H₃BO₃, and HClO₄. In some embodiments, the inorganic acid is selected from HCl, HBr, HI, and H₂SO₄. In some embodiments, the inorganic acid is HBr. In some embodiments, the inorganic acid is HI. In some embodiments, the inorganic acid is H₂SO₄. In some embodiments, the inorganic acid is HCl.

In some embodiments, the ratio of a compound of Formula (III) to the inorganic acid is about 1:1 to about 1:100. In some embodiments, the ratio of a compound of Formula (III) to the inorganic acid is about 1:1, about 1:5, about 1:10, about 1:15, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, about 1:100, about 1:125, about 1:150, about 1:175, about 1:200, about 1:300, about 1:400, or about 1:500. In some embodiments, the ratio of a compound of Formula (III) to the inorganic acid is at least 1:1, at least 1:5, at least 1:10, at least 1:15, at least 1:20, at least 1:25, at least 1:30, at least 1:35, at least 1:40, at least 1:45, at least 1:50, at least 1:55, at least 1:60, at least 1:65, at least 1:70, at least 1:75, at least 1:80, at least 1:85, at least 1:90, at least 1:95, at least 1:100, at least 1:125, at least 1:150, at least 1:175, at least 1:200, at least 1:300, at least 1:400, or at least 1:500. In some embodiments, the ratio of a compound of Formula (III) to the inorganic acid is at most 1:1, at most 1:5, at most 1:10, at most 1:15, at most 1:20, at most 1:25, at most 1:30, at most 1:35, at most 1:40, at most 1:45, at most 1:50, at most 1:55, at most 1:60, at most 1:65, at most 1:70, at most 1:75, at most 1:80, at most 1:85, at most 1:90, at most 1:95, at most 1:100, at most 1:125, at most 1:150, at most 1:175, at most 1:200, at most 1:300, at most 1:400, or at most 1:500. In some embodiments, the ratio of a compound of Formula (III) to HCl is about 1:1 to about 1:100. In some embodiments, the ratio of a compound of Formula (III) to HCl is about 1:1, about 1:5, about 1:10, about 1:15, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, about 1:100, about 1:125, about 1:150, about 1:175, about 1:200, about 1:300, about 1:400, or about 1:500. In embodiments, the ratio of a compound of Formula (III) to HCl is at least 1:1, at least 1:5, at least 1:10, at least 1:15, at least 1:20, at least 1:25, at least 1:30, at least 1:35, at least 1:40, at least 1:45, at least 1:50, at least 1:55, at least 1:60, at least 1:65, at least 1:70, at least 1:75, at least 1:80, at least 1:85, at least 1:90, at least 1:95, at least 1:100, at least 1:125, at least 1:150, at least 1:175, at least 1:200, at least 1:300, at least 1:400, or at least 1:500. In embodiments, the ratio of a compound of Formula (III) to HCl is at most 1:1, at most 1:5, at most 1:10, at most 1:15, at most 1:20, at most 1:25, at most 1:30, at most 1:35, at most 1:40, at most 1:45, at most 1:50, at most 1:55, at most 1:60, at most 1:65, at most 1:70, at most 1:75, at most 1:80, at most 1:85, at most 1:90, at most 1:95, at most 1:100, at most 1:125, at most 1:150, at most 1:175, at most 1:200, at most 1:300, at most 1:400, or at most 1:500.

In some embodiments, the acidifying conditions comprise a solvent. In some embodiments, the solvent is selected from acetone, methanol, ethanol, propanol, isopropanol, n-butanol, isobutanol, tetrahydrofuran, isopropyl acetate, acetonitrile, and methyl ethyl ketone. In some embodiments, the solvent is selected from acetone, isopropyl acetate, acetonitrile, and methyl ethyl ketone. In some embodiments, the solvent is an alcohol. In some embodiments, the alcohol is selected from methanol, ethanol, propanol, isopropanol, n-butanol, and isobutanol. In some embodiments, the solvent is isopropanol (IPA).

In some embodiments, the acidifying conditions comprise an initial concentration of the compound of Formula (III) in the solvent wherein the initial concentration is 0.05 Moles/Liters (mol/L) to about 2 Moles/Liters. In some embodiments, the initial concentration of the compound of Formula (III) in the solvent is about 0.05 mol/L, about 0.10 mol/L, about 0.15 mol/L, about 0.20 mol/L, about 0.25 mol/L, about 0.30 mol/L, about 0.35 mol/L, about 0.40 mol/L, about 0.45 mol/L, about 0.50 mol/L, about 0.55 mol/L, about 0.60 mol/L, about 0.65 mol/L, about 0.70 mol/L, about 0.75 mol/L, about 0.80 mol/L, about 0.85 mol/L, about 0.90 mol/L, about 0.95 mol/L, about 1.00 mol/L, about 1.25 mol/L, about 1.50 mol/L, about 1.75 mol/L, or about 2.00 mol/L. In some embodiments, the initial concentration of the compound of Formula (III) in the solvent is at least 0.05 mol/L, at least 0.10 mol/L, at least 0.15 mol/L, at least 0.20 mol/L, at least 0.25 mol/L, at least 0.30 mol/L, at least 0.35 mol/L, at least 0.40 mol/L, at least 0.45 mol/L, at least 0.50 mol/L, at least 0.55 mol/L, at least 0.60 mol/L, at least 0.65 mol/L, at least 0.70 mol/L, at least 0.75 mol/L, at least 0.80 mol/L, at least 0.85 mol/L, at least 0.90 mol/L, at least 0.95 mol/L, at least 1.00 mol/L, at least 1.25 mol/L, at least 1.50 mol/L, at least 1.75 mol/L, or at least 2.00 mol/L. In some embodiments, the initial concentration of the compound of Formula (III) in the solvent is at most 0.05 mol/L, at most 0.10 mol/L, at most 0.15 mol/L, at most 0.20 mol/L, at most 0.25 mol/L, at most 0.30 mol/L, at most 0.35 mol/L, at most 0.40 mol/L, at most 0.45 mol/L, at most 0.50 mol/L, at most 0.55 mol/L, at most 0.60 mol/L, at most 0.65 mol/L, at most 0.70 mol/L, at most 0.75 mol/L, at most 0.80 mol/L, at most 0.85 mol/L, at most 0.90 mol/L, at most 0.95 mol/L, at most 1.00 mol/L, at most 1.25 mol/L, at most 1.50 mol/L, at most 1.75 mol/L, or at most 2.00 mol/L. In some embodiments, the initial concentration of the compound of Formula (III) in the solvent is between about 0.50 mol/L to about 1.50 mol/L. In some embodiments, the initial concentration of the compound of Formula (III) in the solvent is between about 0.50 mol/L to about 1.25 mol/L. In some embodiments, the initial concentration of the compound of Formula (III) in the solvent is between about 0.50 mol/L to about 1.00 mol/L. In some embodiments, the initial concentration of the compound of Formula (III) in the solvent is between about 0.75 mol/L to about 1.50 mol/L. In some embodiments, the initial concentration of the compound of Formula (III) in the solvent is between about 1.00 mol/L to about 1.50 mol/L. In some embodiments, the initial concentration of the compound of Formula (III) in the solvent is between about 0.75 mol/L to about 1.25 mol/L.

In some embodiments, the acidifying conditions comprise an initial concentration of the compound of Formula (III) in isopropanol wherein the initial concentration is 0.05 Moles/Liters (mol/L) to about 2 Moles/Liters. In some embodiments, the initial concentration of the compound of Formula (III) in isopropanol is about 0.05 mol/L, about 0.10 mol/L, about 0.15 mol/L, about 0.20 mol/L, about 0.25 mol/L, about 0.30 mol/L, about 0.35 mol/L, about 0.40 mol/L, about 0.45 mol/L, about 0.50 mol/L, about 0.55 mol/L, about 0.60 mol/L, about 0.65 mol/L, about 0.70 mol/L, about 0.75 mol/L, about 0.80 mol/L, about 0.85 mol/L, about 0.90 mol/L, about 0.95 mol/L, about 1.00 mol/L, about 1.25 mol/L, about 1.50 mol/L, about 1.75 mol/L, or about 2.00 mol/L. In some embodiments, the initial concentration of the compound of Formula (III) in isopropanol is at least 0.05 mol/L, at least 0.10 mol/L, at least 0.15 mol/L, at least 0.20 mol/L, at least 0.25 mol/L, at least 0.30 mol/L, at least 0.35 mol/L, at least 0.40 mol/L, at least 0.45 mol/L, at least 0.50 mol/L, at least 0.55 mol/L, at least 0.60 mol/L, at least 0.65 mol/L, at least 0.70 mol/L, at least 0.75 mol/L, at least 0.80 mol/L, at least 0.85 mol/L, at least 0.90 mol/L, at least 0.95 mol/L, at least 1.00 mol/L, at least 1.25 mol/L, at least 1.50 mol/L, at least 1.75 mol/L, or at least 2.00 mol/L. In some embodiments, the initial concentration of the compound of Formula (III) in isopropanol is at most 0.05 mol/L, at most 0.10 mol/L, at most 0.15 mol/L, at most 0.20 mol/L, at most 0.25 mol/L, at most 0.30 mol/L, at most 0.35 mol/L, at most 0.40 mol/L, at most 0.45 mol/L, at most 0.50 mol/L, at most 0.55 mol/L, at most 0.60 mol/L, at most 0.65 mol/L, at most 0.70 mol/L, at most 0.75 mol/L, at most 0.80 mol/L, at most 0.85 mol/L, at most 0.90 mol/L, at most 0.95 mol/L, at most 1.00 mol/L, at most 1.25 mol/L, at most 1.50 mol/L, at most 1.75 mol/L, or at most 2.00 mol/L. In some embodiments, the initial concentration of the compound of Formula (III) in isopropanol is between about 0.50 mol/L to about 1.50 mol/L. In some embodiments, the initial concentration of the compound of Formula (III) in isopropanol is between about 0.50 mol/L to about 1.25 mol/L. In some embodiments, the initial concentration of the compound of Formula (III) in isopropanol is between about 0.50 mol/L to about 1.00 mol/L. In some embodiments, the initial concentration of the compound of Formula (III) in isopropanol is between about 0.75 mol/L to about 1.50 mol/L. In some embodiments, the initial concentration of the compound of Formula (III) in isopropanol is between about 1.00 mol/L to about 1.50 mol/L. In embodiments, the initial concentration of the compound of Formula (III) in isopropanol is between about 0.75 mol/L to about 1.25 mol/L.

In some embodiments, the method comprises isolating the compound of Formula (IV). In some embodiments, the isolating is by filtering. In some embodiments, the isolating is by vacuum filtering.

In another aspect, the present disclosure provides a composition comprising a compound represented by (IV-a):

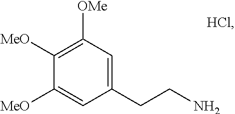 [SMILES: COc1cc(CCN)cc(OC)c1OC ; "HCl,"]

wherein the composition is characterized by one or more additional characteristics selected from: (i) between about 0.1 wt % to about 2 wt % isopropyl alcohol; (ii) between about 0.01 wt % to about 0.1 wt % ethanol; (iii) between about 0.01 wt % to about 0.25 wt % water; (iv) between about 0.1 ppm to about 1 ppm Li; and (v) between about 0.01 ppm to about 0.2 ppm Al. In embodiments, the composition is characterized by two or more characteristics selected from i to v. In embodiments, the composition is characterized by at least two characteristics selected from i to v. In some embodiments, the composition is characterized by at least three characteristics selected from i to v. In embodiments, the composition is characterized by at least four characteristics selected from i to v. In embodiments, the composition is characterized by five characteristics selected from i to v.

In some embodiments, the composition comprises a compound represented by (IV-a):

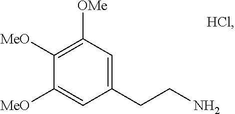 [SMILES: COc1cc(CCN)cc(OC)c1OC ; "HCl,"]

wherein the composition is characterized by one or more additional characteristics selected from: (i) between 0.1 wt % to 2 wt % isopropyl alcohol; (ii) between 0.01 wt % to 0.1 wt % ethanol; (iii) between 0.01 wt % to 0.25 wt % water; (iv) between 0.1 ppm to 1 ppm Li; and (v) between 0.01 ppm to 0.2 ppm Al. In some embodiments, the composition is characterized by two or more characteristics selected from i to v. In some embodiments, the composition is characterized by at least two characteristics selected from i to v. In embodiments, the composition is characterized by at least three characteristics selected from i to v. In embodiments, the composition is characterized by at least four characteristics selected from i to v. In embodiments, the composition is characterized by five characteristics selected from i to v.

In some embodiments, the composition comprises a compound represented by (IV-a):

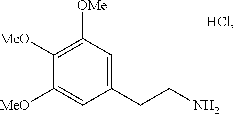 [SMILES: COc1cc(CCN)cc(OC)c1OC ; "HCl,"]

wherein the composition is characterized by one or more additional characteristics selected from: (i) between about 0.1 wt % to about 8.0 wt % 2-(3,4,5-trimethoxyphenyl)acetamide; (ii) between about 0.01 wt % to about 5 wt % 4-(2-aminoethyl)-2,6-dimethoxyphenol; and (iii) between about 0.01 wt % to about 5 wt % 5-(2-aminoethyl)-2,3-dimethoxyphenol. In some embodiments, the composition is characterized by two or more characteristics selected from i to iii. In some embodiments, the composition is characterized by three characteristics selected from i to iii.

In some embodiments, the composition comprises a compound represented by (IV-a):

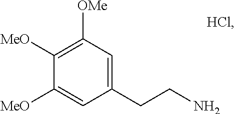 [SMILES: COc1cc(CCN)cc(OC)c1OC ; "HCl,"]

wherein the composition is characterized by one or more additional characteristics selected from: (i) between about 0.1 wt % to about 8.0 wt % 2-(3,4,5-trimethoxyphenyl)acetamide; (ii) between about 0.01 wt % to about 5 wt % 4-(2-aminoethyl)-2,6-dimethoxyphenol; (iii) between about 0.01 wt % to about 5 wt % 5-(2-aminoethyl)-2,3-dimethoxyphenol; and (iv) between about 0.1 wt % to about 5% isopropanol. In some embodiments, the composition is characterized by two or more characteristics selected from i to iv. In some embodiments, the composition is characterized by three or more characteristics selected from i to iv. In some embodiments, the composition is characterized by four characteristics selected from i to iv.

In some embodiments, the composition comprises a compound represented by (IV-a):

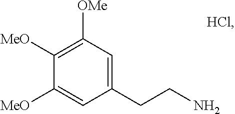 [SMILES: COc1cc(CCN)cc(OC)c1OC ; "HCl,"]

wherein the composition is characterized by one or more additional characteristics selected from: (i) between about 1 ppm to about 50 ppm isopropyl alcohol; (ii) between about 1 ppm to about 50 ppm methanol; (iii) between about 500 ppm to about 1000 ppm ethanol; (iv) between about 0.01 wt % to about 0.25 wt % water; (v) between about 10 wt % to about 20 wt % chloride; (vi) between about 0.1 ppm to about 10 ppm Li; and (vii) between about 0.1 ppm to about 200 ppm Al. In some embodiments, the composition is characterized by two or more characteristics selected from i to vii. In some embodiments, the composition is characterized by three or more characteristics selected from i to vii. In some embodiments, the composition is characterized by four or more characteristics selected from i to vii. In some embodiments, the composition is characterized by five or more characteristics selected from i to vii. In some embodiments, the composition is characterized by six or more characteristics selected from i to vii. In some embodiments, the composition is characterized by seven characteristics selected from i to vii.

In some embodiments, the composition comprises a compound represented by (IV-a):

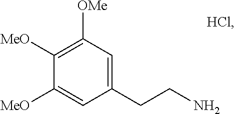 [SMILES: COc1cc(CCN)cc(OC)c1OC ; "HCl,"]

wherein the composition is characterized by one or more additional characteristics selected from: (i) between about 1 ppm to about 50 ppm isopropanol; (ii) between about 1 ppm to about 50 ppm methanol; (iii) between about 500 ppm to about 1000 ppm ethanol; (iv) between about 0.01 wt % to about 0.25 wt % water; (v) between about 10 wt % to about 20 wt % chloride; (vi) between about 0.1 ppm to about 10 ppm Li; (vii) between about 0.1 ppm to about 200 ppm Al; (viii) between about 0.1 wt % to about 8.0 wt % 2-(3,4,5-trimethoxyphenyl)acetamide; (viiii) between about 0.01 wt % to about 5 wt % 4-(2-aminoethyl)-2,6-dimethoxyphenol; and (x) between about 0.01 wt % to about 5 wt % 5-(2-aminoethyl)-2,3-dimethoxyphenol. In some embodiments, the composition is characterized by two or more characteristics selected from i to x. In some embodiments, the composition is characterized by three or more characteristics selected from i to x. In some embodiments, the composition is characterized by four or more characteristics selected from i to x. In some embodiments, the composition is characterized by five or more characteristics selected from i to x. In some embodiments, the composition is characterized by six or more characteristics selected from i to x. In some embodiments, the composition is characterized by seven or more characteristics selected from i to x. In some embodiments, the composition is characterized by eight or more characteristics selected from i to x. In some embodiments, the composition is characterized by nine or more characteristics selected from i to x.

In some embodiments, the composition comprises a compound represented by (IV-a):

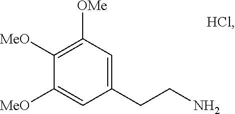 [SMILES: COc1cc(CCN)cc(OC)c1OC ; "HCl,"]

wherein the composition is characterized by one or more additional characteristics selected from: (i) between 0.1 wt % to 8.0 wt % 2-(3,4,5-trimethoxyphenyl)acetamide; (ii) between 0.01 wt % to 5 wt % 4-(2-aminoethyl)-2,6-dimethoxyphenol; and (iii) between 0.01 wt % to 5 wt % 5-(2-aminoethyl)-2,3-dimethoxyphenol. In some embodiments, the composition is characterized by two or more characteristics selected from i to iii. In some embodiments, the composition is characterized by three characteristics selected from i to iii.

In some embodiments, the composition comprises a compound represented by (IV-a):

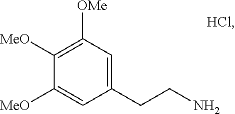 [SMILES: COc1cc(CCN)cc(OC)c1OC ; "HCl,"]

wherein the composition is characterized by one or more additional characteristics selected from: (i) between 0.1 wt % to 8.0 wt % 2-(3,4,5-trimethoxyphenyl)acetamide; (ii) between 0.01 wt % to 5 wt % 4-(2-aminoethyl)-2,6-dimethoxyphenol; (iii) between 0.01 wt % to 5 wt % 5-(2-aminoethyl)-2,3-dimethoxyphenol; and (iv) between 0.1 wt % to 5% isopropanol. In some embodiments, the composition is characterized by two or more characteristics selected from i to iv. In some embodiments, the composition is characterized by three or more characteristics selected from i to iv. In some embodiments, the composition is characterized by four characteristics selected from i to iv.

In some embodiments, the composition comprises a compound represented by (IV-a):

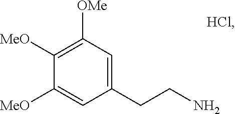 [SMILES: COc1cc(CCN)cc(OC)c1OC ; "HCl,"]

wherein the composition is characterized by one or more additional characteristics selected from: (i) between 1 ppm to 50 ppm isopropyl alcohol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; and (vii) between 0.1 ppm to 200 ppm Al. In some embodiments, the composition is characterized by two or more characteristics selected from i to vii. In some embodiments, the composition is characterized by three or more characteristics selected from i to vii. In some embodiments, the composition is characterized by four or more characteristics selected from i to vii. In some embodiments, the composition is characterized by five or more characteristics selected from i to vii. In some embodiments, the composition is characterized by six or more characteristics selected from i to vii. In some embodiments, the composition is characterized by seven characteristics selected from i to vii.

In some embodiments, the composition comprises a compound represented by (IV-a):

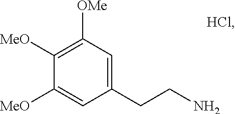 [SMILES: COc1cc(CCN)cc(OC)c1OC ; "HCl,"]

wherein the composition is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (viiii) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the composition is characterized by two or more characteristics selected from i to x. In some embodiments, the composition is characterized by three or more characteristics selected from i to x. In some embodiments, the composition is characterized by four or more characteristics selected from i to x. In some embodiments, the composition is characterized by five or more characteristics selected from i to x. In some embodiments, the composition is characterized by six or more characteristics selected from i to x. In some embodiments, the composition is characterized by seven or more characteristics selected from i to x. In some embodiments, the composition is characterized by eight or more characteristics selected from i to x. In some embodiments, the composition is characterized by nine or more characteristics selected from i to x.

### Pharmaceutical Formulations

In some aspects, provided herein are compositions, such as pharmaceutical compositions, comprising the disclosed compounds, such as compounds of Formula (IV). While it is possible to administer a compound employed in the disclosed methods directly without any formulation, the compounds are usually administered in the form of pharmaceutical compositions.

“Pharmaceutical compositions” are compositions that include the disclosed compound(s) together in an amount (for example, in a unit dosage form) with a pharmaceutically acceptable carrier, diluent, or excipient. Some embodiments will not have a single carrier, diluent, or excipient alone, but will include multiple carriers, diluents, and/or excipients. Compositions can be prepared by standard pharmaceutical formulation techniques such as disclosed in, e.g., Remington: The Science & Practice of Pharmacy (2020) 23th ed., Academic Press., Cambridge, Mass.; The Merck Index (1996) 12th ed., Merck Pub. Group, Whitehouse, N.J.; Pharm. Principles of Solid Dosage Forms (1993), Technomic Pub. Co., Inc., Lancaster, Pa.; and Ansel & Stoklosa, Pharm. Calculations (2001) 11th ed., Lippincott Williams & Wilkins, Baltimore, Md.; & Poznansky et al. Drug Delivery Systems (1980), R. L. Juliano, ed., Oxford, N.Y, pp. 253-315).

“Pharmaceutically acceptable” used in connection with an excipient, carrier, diluent, or other ingredient means the ingredient is generally safe and, within the scope of sound medical judgment, suitable for use in contact with cells of humans and animals without undue toxicity, irritation, allergic response, or complication, commensurate with a reasonable risk/benefit ratio.

In certain aspects, the present disclosure provides a pharmaceutical composition comprising at least one pharmaceutically acceptable excipient and a compound or salt of Formula (IV). The composition may be in the form of a solid, liquid, gel, semi-liquid, or semi-solid. Pharmaceutical compositions of the disclosure suitable for oral administration can be presented as discrete dosage forms, such as hard or soft capsules, cachets, troches, lozenges, or tablets, or liquids or aerosol sprays. The compounds are, in some embodiments, formulated into suitable preparations such as solutions, suspensions, tablets, dispersible tablets, pills, capsules, powders, sustained release formulations or elixirs, for oral administration or in sterile solutions or suspensions for parenteral administration, transdermal administration and oral inhalation via nebulizers, pressurized metered dose inhalers and dry powder inhalers. In some embodiments, mescaline or a salt thereof may be formulated into compositions using techniques and procedures well known in the art (see, e.g., Ansel, Intro. to Pharmaceutical Dosage Forms, 7th Ed. (1999)).

In some embodiments, the pharmaceutical composition is formulated into a pharmaceutical formulation. Pharmaceutical formulations may be provided in any suitable form, which may depend on the route of administration. In some embodiments, the pharmaceutical composition disclosed herein can be formulated in dosage form for administration to a subject.

In some embodiments, the pharmaceutical composition is formulated for oral, sublingual intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments parenteral administration characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions.

In making the compositions employed in the invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, microemulsions, liposomal dispersions, aqueous and non-aqueous solutions, slurries, syrups, aerosols (as a solid or in a liquid medium), ointments containing for example up to 10% by weight of the active compound, soft and hard gelatin capsules, suppositories, topical preparations, transdermal patches, sterile injectable solutions, and sterile packaged powders. Compositions may be formulated as immediate release, controlled release, sustained (extended) release or modified release formulations. In some embodiments, the composition is prepared as a dry powder for inhalation or a liquid preparation for vaporization and inhalation, and is administered, e.g., using an electronic cigarette or other vaping device, a nebulizer, a pressurized metered dose inhaler (pMDI), a dry powder inhaler (DPI), or the like.

Different embodiments of the invention include the following examples: Pharmaceutically acceptable complex derivatives of each drug in each group, including solvates, salts, esters, enantiomers, isomers (stereoisomers and/or constitutional, including ones based on substituting fluorine for hydrogen), derivatives or prodrugs of the disclosed compounds. Among derivatives of a compound are included its “physiologically functional derivatives,” which refers to physiologically tolerated chemical derivatives of the compound having the same physiological function thereof, for example, by being convertible in the body thereto, and which on administration to a mammal such as a human is able to form (directly or indirectly) the compound or an active metabolite thereof (acting therefore, like a prodrug), or by otherwise having the same physiological function, despite one or more structural differences. According to the present invention, examples of physiologically functional derivatives include esters, amides, carbamates, ureas, and heterocycles.

In other embodiments are disclosed multiple variations in the pharmaceutical dosages of disclosed compounds as further outlined below. Another embodiment of the invention includes various forms of preparations including using solids, liquids, immediate or delayed or extended-release forms. Many types of variations are possible as known to those of skill.

In other embodiments are disclosed multiple routes of administration, which may differ in different patients according to their preference, comorbidities, side effect profile, pharmacokinetic and pharmacodynamic considerations, and other factors (IV, PO, transdermal, etc.). In other embodiments are disclosed the presence of other substances with the active drugs, known to those of skill, such as fillers, carriers, gels, skin patches, lozenges, or other modifications in the preparation to facilitate absorption through various routes (such as gastrointestinal, transdermal, ocular, intraocular, etc.) and/or to extend the effect of the drugs, and/or to attain higher or more stable serum levels or to enhance the therapeutic effect of the disclosed compounds.

In preparing a formulation, it may be necessary to mill a disclosed compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh.

Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. Formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The disclosed compositions can be formulated so as to provide quick, sustained or delayed release of the active agent(s) after administration to the patient by employing procedures known in the art.

The disclosed compositions are preferably formulated in a unit dosage form, each dosage containing a therapeutically effective amount of the active ingredients, for example in the dosage amounts disclosed below. The term “unit dosage form” refers to a physically discrete unit suited as unitary dosages for the subject to be treated, each unit containing a predetermined quantity of active material calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose for a “booster” dose as described below), of the pharmaceutical composition administered.

Unit dosage forms include capsules, troches, cachets, lozenges, tablets, ampules and vials, which may include a composition in a freeze-dried or lyophilized state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis (including the mucosa) of a subject for an extended or brief period of time.

In embodiments, compositions comprising a compound provided herein are formulated in a pharmaceutically acceptable oral dosage form, including oral solid dosage forms and oral liquid dosage forms. In some embodiments, the compositions are formulated as a pharmaceutically acceptable oral solid dosage form. Oral solid dosage forms may include but are not limited to, lozenges, troches, tablets, capsules, caplets, powders, pellets, multiparticulates, beads, spheres, and/or any combinations thereof. Oral solid dosage forms may be formulated as immediate release, controlled release, sustained release, extended release, or modified release formulations.

In some embodiments, the solid dosage provided herein, such as oral solid dosage forms, may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived IPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. In some embodiments, pharmaceutical formulations described herein may be administered as a single capsule or in multiple capsule dosage form. In some embodiments, the pharmaceutical formulation is administered in two, three, four, or more capsules or tablets.

In some embodiments, solid dosage forms comprise pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. In embodiments, solid dosage forms comprise one or more pharmaceutically acceptable additives such as a compatible carrier, complexing agent, ionic dispersion modulator, disintegrating agent, surfactant, lubricant, colorant, moistening agent, plasticizer, stabilizer, wetting agent, anti-foaming agent, alone or in combination, as well as supplementary active agent(s). In some embodiments, supplementary active agents include preservatives, antioxidants, antimicrobial agents including biocides and biostats such as antibacterial, antiviral and antifungal agents.

In some aspects, provided herein are methods for preparing a composition, such as a pharmaceutical composition comprising a compound described herein. In some embodiments, a pharmaceutical composition, as provided herein, comprises one or more excipients, such as a pharmaceutically acceptable excipient. Non-limiting examples of excipients include fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the pharmaceutical composition may be an immediate release formulation, wherein a therapeutically effective amount of the pharmaceutical composition is administered to the subject in a way that facilitates rapid release. Immediate-release formulations may be prepared by combining a superdisintegrant such as croscarmellose sodium and different grades of microcrystalline cellulose in different ratios. In some embodiments, to aid disintegration, sodium starch glycolate may be added.

In embodiments, tablets provided herein are prepared by methods well known in the art. Various methods for the preparation of the immediate release, modified release, controlled release, and extended-release dosage forms (e.g., as matrix tablets having one or more modified, controlled, or extended-release layers) and the vehicles therein are well known in the art. In embodiments, a tablet may be made by compression or molding. In embodiments, compressed tablets may be prepared by compressing, in a suitable machine, an active ingredient in a free-flowing form such as a powder or granules, optionally mixed with a binder, lubricant, inert diluent, preservative, surface-active or dispersing agent. In embodiments, molded tablets may be produced by molding, in a suitable apparatus, a mixture of powdered compound moistened with an inert liquid diluent. In some embodiments, the tablets may optionally be coated or scored and may be formulated so as to provide a slow or controlled release of the active ingredient therein. Generally recognized compendia of methods include Remington 2020 and Sheth et al. 1980.

In embodiments, solid dosage forms are prepared by mixing the active agents of the invention with one or more pharmaceutical excipients to form a “bulk blend” composition. In some embodiments, the bulk blend composition is homogeneous, i.e., the active agents are dispersed evenly throughout so that the bulk blend may be readily subdivided into equally effective unit dosage forms, such as tablets, pills, and capsules. In embodiments, the individual unit dosages may also comprise film coatings, which disintegrate upon oral ingestion or upon contact with diluents. In embodiments, these formulations are manufactured by conventional pharmaceutical techniques. Conventional pharmaceutical techniques for preparation of solid dosage forms include, but are not limited to, the following methods, which may be used alone or in combination: (1) dry mixing, (2) direct compression, (3) milling, (4) dry or non-aqueous granulation, (5) wet granulation, or (6) fusion (see, e.g., Lachman et al. 1986). Other methods include spray drying, pan coating, melt granulation, granulation, fluidized bed spray drying or coating (e.g., Wurster coating), tangential coating, top spraying, tableting, and extruding.

In some embodiments, a composition comprising a compound as provided herein can be formulated to achieve a specific release profile. In some embodiments, oral solid dosage forms may be prepared as immediate release formulations, or as modified release formulations, such as controlled release, extended release, sustained release, or delayed release.

In some embodiments, a composition comprising a compound provided herein is formulated as a pharmaceutically acceptable oral liquid dosage form. Non-limiting examples of oral liquid dosage forms include tinctures, drops, emulsions, syrups, elixirs, suspensions, and solutions, and the like. In some embodiments, oral liquid dosage forms may be formulated with any pharmaceutically acceptable excipient known to those of skill for the preparation of liquid dosage forms, and with solvents, diluents, carriers, excipients, and the like, chosen as appropriate to the solubility and other properties of the active agents and other ingredients. Non-limiting examples of solvents include, e.g., water, glycerin, simple syrup, alcohol, medium chain triglycerides (MCT), and combinations thereof.

In some embodiments, oral liquid dosage forms may be monophasic or biphasic, the former being a substantially homogenous solution dissolved in water or non-aqueous solvent, while the latter refers to oral liquid dosage forms in which the active ingredients do not fully dissolve in common solvents. In some embodiments, over time, the solid particles (i.e., the active agents) within the oral liquid dosage form may form a precipitate at the bottom of the container-requiring vigorous shaking to redisperse the active ingredients. Non-limiting examples of monophasic liquid forms include syrups, linctuses, spirits/essences, elixirs, and fluid extracts. Non-limiting examples of biphasic liquid forms include oral suspensions, oral emulsions, and mixtures.

Liquid dosage forms for oral administration may be prepared as liquid suspensions or solutions using a sterile liquid, such as but not limited to, an oil, water, an alcohol, combinations of pharmaceutically suitable surfactants, suspending agents, and emulsifying agents. In some embodiments, liquid formulations also may be prepared as single dose or multi-dose beverages. In some embodiments, suspensions may include oils. Such oils include but are not limited to peanut oil, sesame oil, cottonseed oil, corn oil, and olive oil. Suitable oils also include carrier oils such as MCT and long chain triglyceride (LCT) oils. In some embodiments, as suspension preparation may also contain esters of fatty acids such as ethyl oleate, isopropyl myristate, fatty acid glycerides, and acetylated fatty acid glycerides. In some embodiments, suspension formulations may include alcohols, such as ethanol, isopropyl alcohol, hexadecyl alcohol; glycerol, and propylene glycol. In some embodiments, ethers, such as polyethylene glycol; petroleum hydrocarbons, such as mineral oil and petrolatum; and water may also be used in suspension formulations. In some embodiments, a suspension can thus include an aqueous liquid or a non-aqueous liquid, an oil-in-water liquid emulsion, or a water-in-oil emulsion.

Dosage forms for oral administration may be aqueous suspensions such as aqueous oral dispersions, emulsions, solutions, and syrups (see, e.g., Singh et al., Encyclopedia of Pharm. Technology, 2nd Ed., 751-753, 2002). In addition to the active agents, the liquid dosage forms may comprise additives, such as one or more (a) disintegrating agents, (b) dispersing agents, (c) wetting agents, (d) preservatives, (e) viscosity enhancing agents, (f) sweetening agents, and/or (g) flavoring agents. In addition to the additives above, the liquid formulations of the invention, in some embodiments, may also comprise inert diluents commonly used in the art such as water or other solvents, solubilizing agents, emulsifiers, flavoring agents, and/or sweeteners. In some embodiments, co-solvents and adjuvants also may be added to a formulation.

In some embodiments, effervescent powders containing the compositions of the invention may be prepared. In some embodiments, effervescent salts are used to disperse medicines in water for oral administration. In some embodiments, effervescent salts also may be packaged as single dose or multi-dose drink mixes, alone or in combination with other ingredients, such as vitamins or electrolytes. In some embodiments, effervescent salts are granules or coarse powders containing a medicinal agent in a dry mixture, usually composed of sodium bicarbonate and sodium carbonate, citric acid, and/or tartaric acid. In some embodiments, when salts of the invention are added to water, the acids and the base react to liberate carbon dioxide gas, thereby causing “effervescence.” In some embodiments, any acid-base combination that results in the liberation of carbon dioxide may be used, as long as the ingredients are suitable for pharmaceutical use, and result in a pH of about 6.0 or higher.

In some embodiments, the pharmaceutical composition disclosed herein can be formulated for sublingual administration. In some embodiments, the disclosure provides a pharmaceutical composition for oral administration comprising a compound or salt of Formula (IV) and a pharmaceutical excipient suitable for oral administration.

In some embodiments, the pharmaceutical composition comprises from 50-2000 mg of a compound or salt of Formula (IV). In some embodiments, the pharmaceutical composition comprises from about 50-1500 mg of mescaline or salt thereof. In some embodiments, the pharmaceutical composition comprises from about 50-1000 mg of mescaline or salt thereof. In some embodiments, the pharmaceutical composition comprises from about 50-900 mg of mescaline or salt thereof. In some embodiments, the pharmaceutical composition comprises from about 50-800 mg of mescaline or salt thereof. In some embodiments, the pharmaceutical composition comprises from about 50-700 mg of mescaline or salt thereof. In some embodiments, the pharmaceutical composition comprises from about 50-600 mg of mescaline or salt thereof. In some embodiments, the pharmaceutical composition comprises from about 50-500 mg of a compound or salt of Formula (IV). In some embodiments, the pharmaceutical composition comprises from about 50-400 mg of a compound or salt of Formula (IV). In some embodiments, the pharmaceutical composition comprises from about 50-300 mg of a compound or salt of Formula (IV). In some embodiments, the pharmaceutical composition comprises from about 50-200 mg of a compound or salt of Formula (IV). In some embodiments, the pharmaceutical composition comprises from about 200-1000 mg of a compound or salt of Formula (IV). In some embodiments, the pharmaceutical composition comprises from about 300-1000 mg of a compound or salt of Formula (IV). In some embodiments, the pharmaceutical composition comprises from about 400-1000 mg of a compound or salt of Formula (IV). In some embodiments, the pharmaceutical composition comprises from about 500-1000 mg of a compound or salt of Formula (IV). In some embodiments, the pharmaceutical composition comprises from about 600-1000 mg of a compound or salt of Formula (IV). In some embodiments, the pharmaceutical composition comprises about 50 mg, about 100 mg, about 200 mg, about 300 mg, about 400 mg, about 500 mg, about 600 mg, about 700 mg, about 800 mg, about 900 mg, about 1000 mg, about 1500 mg, or about 2000 mg of a compound or salt of Formula (IV). In some embodiments, the pharmaceutical composition comprises at least 50 mg, at least 100 mg, at least 200 mg, at least 300 mg, at least 400 mg, at least 500 mg, at least 600 mg, at least 700 mg, at least 800 mg, at least 900 mg, at least 1000 mg, at least 1500 mg, or at least 2000 mg of a compound or salt of Formula (IV). In some embodiments, the pharmaceutical composition comprises at most 50 mg, at most 100 mg, at most 200 mg, at most 300 mg, at most 400 mg, at most 500 mg, at most 600 mg, at most 700 mg, at most 800 mg, at most 900 mg, at most 1000 mg, at most 1500 mg, or at most 2000 mg of a compound or salt of Formula (IV).

In some aspects, the compounds or salts thereof, described herein are useful for the treatment of substance use disorders. In some embodiments, the method comprises administering to a patient suffering from a substance use disorder a composition comprising: a compound represented by (IV-a):

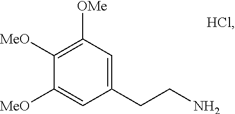 [SMILES: COc1cc(CCN)cc(OC)c1OC ; "HCl,"]

wherein the composition is characterized by one or more additional characteristics selected from: (i) between about 0.1 wt % to about 2 wt % isopropyl alcohol; (ii) between about 0.01 wt % to about 0.1 wt % ethanol; (iii) between about 0.01 wt % to about 0.25 wt % water; (iv) between about 0.1 ppm to about 1 ppm Li; and (v) between 0.01 ppm to about 0.2 ppm Al.

In some embodiments, the compounds described herein may be used to treat a substance use disorder or alcohol use disorder as described in U.S. Provisional Application 63/194,863, U.S. Provisional Application 63/270,989, and WO2022/251690, the entire contents of each of which are incorporated by reference.

### ENUMERATED EMBODIMENTS

Embodiment 1A. A method for synthesizing a compound, the method comprising contacting a compound represented by (equivalently as shorthand, “compound of”) Formula (I):

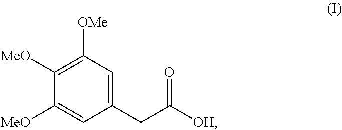 [SMILES: COc1cc(CC(=O)O)cc(OC)c1OC]

with a coupling agent and under amination conditions, wherein the by-product of the contacting is CO₂, to produce a compound of Formula (II):

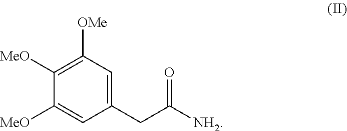 [SMILES: COc1cc(CC(N)=O)cc(OC)c1OC]

Embodiment 2A. The method of embodiment 1A, wherein the amination conditions comprise a first solvent selected from a polar aprotic solvent.

Embodiment 3A. The method of embodiment 2A, wherein the polar aprotic solvent selected from acetone, acetonitrile, dichloromethane, chloroform, dimethylformamide, dimethylsulfoxide, ethyl acetate, and tetrahydrofuran, dimethyl ether, diethyl ether, dioxane, pyran, furan, dimethoxyethane, methyl tert-butyl ether, and chlorobenzene.

Embodiment 4A. The method of embodiment 3A, wherein the polar aprotic solvent is selected from dimethyl ether, diethyl ether, tetrahydrofuran, and dimethoxyethane.

Embodiment 5A. The method of embodiment 4A, wherein the polar aprotic solvent is tetrahydrofuran.

Embodiment 6A. The method of any one of embodiments 1A to 5A, wherein the amination conditions comprise an amination reagent.

Embodiment 7A. The method of embodiment 6A, wherein the amination reagent is ammonia.

Embodiment 8A. The method of embodiment 7A, wherein the amination conditions comprise sparging ammonia in the first solvent.

Embodiment 9A. The method of embodiment 8A, wherein the sparging ammonia comprises a sparge time of about 5 minutes to about 60 minutes.

Embodiment 10A. The method of any one of embodiments 1A to 9A, wherein the coupling agent is carbonyldiimidazole.

Embodiment 11A. The method of any one of embodiments 1A to 10A, wherein the amination conditions comprise maintaining a first reaction temperature of about 15° C. to about 50° C.

Embodiment 12A. The method of claim of any one of embodiments 1A to 11A, wherein the mole ratio of the compound of Formula (I) to the coupling agent is about 1:1 to about 1:5.

Embodiment 13A. The method of any of embodiments 1A to 12A, wherein the amination conditions comprise a first reaction time from about 15 minutes to about 250 minutes.

Embodiment 14A. The method of any one of embodiments 1A to 13A, wherein the concentration of the compound of Formula (I) in the first solvent is about 0.05 Moles/Liters to about 2 Moles/Liters prior to the addition of the coupling agent or the amination conditions.

Embodiment 15A. The method of any one of embodiments 1A to 14A, wherein the method further comprises:
contacting the compound of Formula (II) under reducing conditions, to produce a compound of Formula (III):

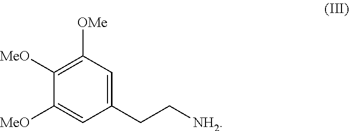 [SMILES: COc1cc(CCN)cc(OC)c1OC]

Embodiment 16A. The method of embodiment 15A, wherein the reducing conditions comprise a reducing agent.

Embodiment 17A. The method of embodiment 16A, wherein the reducing agent is a hydrogen donor, hydrogen, or a metal hydride.

Embodiment 18A. The method of embodiment 16A, wherein the reducing agent is selected from LiAlH₄, hydrogen, NaBH₄, di-isobutyl aluminum hydride (DIBAL-H), oxalic acid, and formic acid.

Embodiment 19A. The method of embodiment 18A, wherein the reducing agent is LiAlH₄.

Embodiment 20A. The method of embodiment 19A, wherein the mole ratio of the compound of Formula (I) to LiAlH₄ to is about 1:1 to about 1:5.

Embodiment 21A. The method of any one of embodiments 16A to 20A, wherein the reducing conditions further comprise a metal halide.

Embodiment 22A. The method of embodiment 21A, wherein the reducing conditions comprise AlCl₃.

Embodiment 23A. The method of embodiment 22A, wherein the mole ratio of LiAlH₄ to AlCl₃ is from 4:1 to 1:4.

Embodiment 24A. The method of any one of embodiments 16A to 23A, wherein the reducing conditions comprise a second solvent.

Embodiment 25A. The method of embodiment 24A, wherein the second solvent is a polar aprotic solvent selected from acetone, acetonitrile, dichloromethane, chloroform, dimethylformamide, dimethylsulfoxide, ethyl acetate, and tetrahydrofuran, dimethyl ether, diethyl ether, dioxane, pyran, furan, dimethoxyethane, methyl tert-butyl ether, and chlorobenzene.

Embodiment 26A. The method of embodiment 24A, wherein the polar aprotic solvent is selected from dimethyl ether, diethyl ether, tetrahydrofuran, and dimethoxyethane.

Embodiment 27A. The method of embodiment 26A, wherein the polar aprotic solvent is tetrahydrofuran.

Embodiment 28A. The method of embodiment 16A, wherein the reducing conditions comprise a second reaction temperature.

Embodiment 29A. The method of embodiment 28A, wherein the second reaction temperature is from about 35° C. to about 100° C.

Embodiment 30A. The method of embodiment 29A, wherein the reducing conditions comprise refluxing.

Embodiment 31A. The method of any one of embodiments 1A to 30A, wherein the method further comprises:
contacting the compound of Formula (III) with an acid under acidifying conditions to produce a compound of Formula (IV):

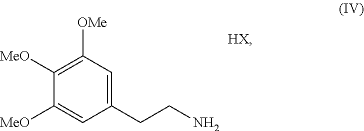

wherein X represents the conjugate base of the acid.

Embodiment 32A. The method of embodiment 31A, wherein the acid is an inorganic acid.

Embodiment 33A. The method of embodiment 32A, wherein the inorganic acid is selected from: HCl, HBr, HI, HF, HNO₃, H₃PO₄, H₂SO₄, H₃BO₃, and HClO₄.

Embodiment 34A. The method of embodiment 33A, wherein the inorganic acid is selected from HCl, HBr, HI, and H₂SO₄.

Embodiment 35A. The method of embodiment 34A, wherein the inorganic acid is HCl.

Embodiment 36A. The method of embodiment 35A, wherein the ratio of a compound of Formula (III) to the inorganic acid is about 1:1 to about 1:100.

Embodiment 37A. The method of any one of embodiments 32A to 36A, wherein the acidifying conditions comprise a third solvent.

Embodiment 38A. The method of embodiment 37A, wherein the third solvent is selected from acetone, methanol, ethanol, propanol, isopropanol, n-butanol, isobutanol, tetrahydrofuran, isopropyl acetate, acetonitrile, and methyl ethyl ketone.

Embodiment 39A. The method of embodiment 38A, wherein the third solvent is isopropanol.

Embodiment 40A. The method of any one of embodiments 31A to 39A, wherein the acidifying conditions comprise an initial concentration of the compound of Formula (III) in the third solvent wherein the initial concentration is 0.05 Moles/Liters to about 2 Moles/Liters.

Embodiment 41A. The method of embodiment 40A, wherein the method further comprises isolating the compound of Formula (IV) by filtering.

Embodiment 42A. The method of any one of the preceding embodiments, wherein the method is performed under Good Manufacturing Practices (GMP) conditions.

Embodiment 43A. The method of any one of the preceding embodiments, wherein the method is performed in a GMP facility.

Embodiment 44A. A composition comprising a compound of Formula (II):

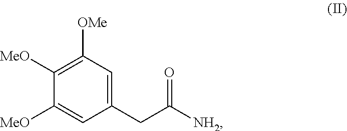 [SMILES: COc1cc(CC(N)=O)cc(OC)c1OC]

wherein the composition is characterized by one or more additional characteristic selected from:
i. between about 0.1 wt % to about 5 wt % imidazole;
ii. between about 0.1 wt % to about 1.0 wt % dichloromethane; and
iii. between about 0.1 wt % to about 3.0 wt % 3,4,5-Trimethoxyphenyl acetic acid.

Embodiment 45A. The composition of embodiment 44A, wherein the compositions is characterized by at least two characteristics selected from i to iii.

Embodiment 46A. A composition comprising a compound of Formula (III):

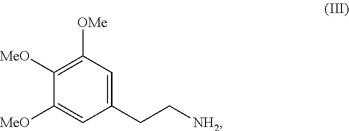 [SMILES: COc1cc(CCN)cc(OC)c1OC]

wherein the composition is characterized by one or more additional characteristic selected from:
i. between about 0.1 wt % to about 8.0 wt % 2-(3,4,5-trimethoxyphenyl)acetamide;
ii. between about 0.1 wt % to about 5 wt % dimeric amine;
iii. between about 0.1 wt % to about 5 wt % n-butanol; and
iv. between about 0.1 wt % to about 6% toluene.

Embodiment 47A. The composition of embodiment 46A, wherein the composition is characterized by two or more characteristics selected from i to iv.

Embodiment 48A. The composition of embodiment 46A or 47A, wherein the composition is characterized by at least two characteristics selected from i to iv.

Embodiment 49A. The composition of any one of embodiments 46A to 48A, wherein the composition is characterized by at least three characteristics selected from i to iv.

Embodiment 50A. The composition of any one of embodiments 46A to 49A, wherein the composition is characterized by four characteristics selected from i to iv.

Embodiment 51A. A composition comprising a compound of Formula (IV-a):

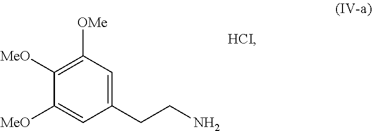 [SMILES: COc1cc(CCN)cc(OC)c1OC ; "HCI,"]

wherein the composition is characterized by one or more additional characteristics selected from:
i. between about 0.1 wt % to about 2 wt % isopropyl alcohol;
ii. between about 0.01 wt % to about 0.1 wt % ethanol;
iii. between about 0.01 wt % to about 0.25 wt % water;
iv. between about 0.1 ppm to about 1 ppm Li; and
v. between 0.01 ppm to about 0.2 ppm Al.

Embodiment 52A. The composition of embodiment 51A wherein the composition is characterized by at least two characteristics selected from i to v.

Embodiment 53A. The composition of embodiment 51A or 52A, wherein the composition is characterized by at least two characteristics selected from i to v.

Embodiment 54A. The composition of any one of claims 51A to 53A, wherein the composition is characterized by at least three characteristics selected from i to v.

Embodiment 55A. The composition of any one of embodiments 51A to 54A, wherein the composition is characterized by at least four characteristics selected from i to v.

Embodiment 56A. The composition of any one of embodiments 51A to 55A, wherein the composition is characterized by five characteristics selected from i to v.

Embodiment 57A. A composition comprising a compound of Formula (IV-a):

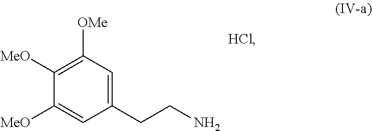 [SMILES: COc1cc(CCN)cc(OC)c1OC ; "HCl,"]

wherein the composition is characterized by one or more additional characteristics selected from:
i. between about 0.1 wt % to about 8.0 wt % 2-(3,4,5-trimethoxyphenyl)acetamide;
ii. between about 0.01 wt % to about 5 wt % 4-(2-aminoethyl)-2,6-dimethoxyphenol; and
iii. between about 0.01 wt % to about 5 wt % 5-(2-aminoethyl)-2,3-dimethoxyphenol;

Embodiment 58A. The composition of claim 57A, wherein the composition is characterized by at least two characteristics selected from i to iii.

Embodiment 59A. The composition of embodiment 57A or 58A, wherein the composition is characterized by three characteristics selected from i to ii.

Embodiment 60A. A composition comprising a compound of Formula (IV-a):

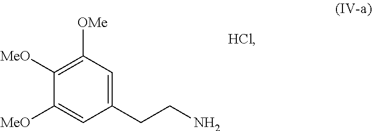 [SMILES: COc1cc(CCN)cc(OC)c1OC ; "HCl,"]

wherein the composition is characterized by one or more additional characteristics selected from:
(i) between 1 ppm to 50 ppm isopropanol;
(ii) between 1 ppm to 50 ppm methanol;
(iii) between 500 ppm to 1000 ppm ethanol;
(iv) between 0.01 wt % to 0.25 wt % water;
(v) between 10 wt % to 20 wt % chloride;
(vi) between 0.1 ppm to 10 ppm Li;
(vii) between 0.1 ppm to 200 ppm Al;
(viii) between 0.1 wt % to 8.0 wt % 2-(3,4,5-trimethoxyphenyl)acetamide;
(ix) between 0.01 wt % to 5 wt % 4-(2-aminoethyl)-2,6-dimethoxyphenol; and
(x) between 0.01 wt % to 5 wt % 5-(2-aminoethyl)-2,3-dimethoxyphenol

Embodiment 61A. The composition of embodiment 60A, wherein the composition is characterized by at least two characteristics selected from i to x.

Embodiment 62A. The composition of embodiment 60A or 61A, wherein the composition is characterized by at least three characteristics selected from i to x.

Embodiment 63A. The composition of any one of embodiments 60A to 62A, wherein the composition is characterized by at least four characteristics selected from i to x.

Embodiment 64A. The composition of any one of embodiments 60A to 63A, wherein the composition is characterized by at least five characteristics selected from i to x.

Embodiment 65A. The composition of any one of embodiments 60A to 64A, wherein the composition is characterized by at least six characteristics selected from i to x.

Embodiment 66A. The composition of any one of embodiments 60A to 65A, wherein the composition is characterized by at least seven characteristics selected from i to x.

Embodiment 67A. The composition of any one of embodiments 60A to 66A, wherein the composition is characterized by at least eight characteristics selected from i to x

Embodiment 68A. The composition of any one of embodiments 60A to 67A, wherein the composition is characterized by at least nine characteristics selected from i to x

Embodiment 69A. The composition of any one of embodiments 60A to 68A, wherein the composition is characterized by ten characteristics selected from i to x

Embodiment 70A. A pharmaceutical composition, comprising a composition of any one of embodiments 44 to 69 and a pharmaceutically acceptable excipient.

Embodiment 71A. A method of treating a substance abuse disorder, comprising administering a composition of any one of embodiments 44A to 69A or a pharmaceutical composition of embodiment 70A to a subject in need thereof.

Embodiment 1B. A method of preparing a compound of Formula (III):

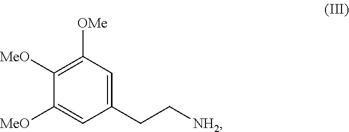 [SMILES: COc1cc(CCN)cc(OC)c1OC]

or a salt thereof, comprising: (i) subjecting a compound of Formula (I):

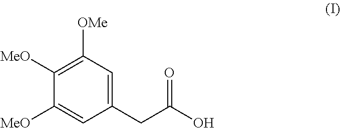 [SMILES: COc1cc(CC(=O)O)cc(OC)c1OC]

to amination conditions to form a compound of Formula (II):

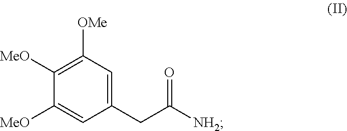 [SMILES: COc1cc(CC(N)=O)cc(OC)c1OC]

and (ii) subjecting the compound of Formula (II) to reducing conditions to produce the compound of Formula (III), or a salt thereof.

Embodiment 2B. The method of embodiment 1B, wherein step (i) is conducted in a polar aprotic solvent.

Embodiment 3B. The method of embodiment 2B, wherein the polar aprotic solvent selected from the group consisting of acetone, acetonitrile, dichloromethane, chloroform, dimethylformamide, dimethylsulfoxide, ethyl acetate, tetrahydrofuran, diethyl ether, dioxane, furan, dimethoxyethane, methyl tert-butyl ether, and chlorobenzene.

Embodiment 4B. The method of embodiment 3B, wherein the polar aprotic solvent is tetrahydrofuran.

Embodiment 5B. The method of embodiment 1B, wherein the amination conditions comprise an amination reagent.

Embodiment 6B. The method of embodiment 5B, wherein the amination reagent is ammonia.

Embodiment 7B. The method of embodiment 5B, further comprising a coupling agent.

Embodiment 8B. The method of embodiment 7B, wherein the coupling agent is selected from the group consisting of dicyclohexylcarbodiimide (DCC), diisopropylcarbodiimide (DIC), 1-ethyl-3-(3-dimethylaminopropyl)carbodiimide (EDC), benzotriazol-1-yloxytris(dimethylamino)phosphonium hexafluorophosphate (BOP), 1-[bis(dimethylamino)methylene]-1H-1,2,3-triazolo[4,5-b]pyridinium 3-oxide hexafluorophosphate (HATU), carbonyldiimidazole (CDI), and carbonylditriazole.

Embodiment 9B. The method of embodiment 8B, wherein the coupling agent is carbonyldiimidazole (CDI).

Embodiment 10B. The method of embodiment 7B, wherein the molar ratio of the compound of Formula (I) to the coupling agent is between about 1:1 and 1:5.

Embodiment 11B. The method of embodiment 10B, wherein the molar ratio of the compound of Formula (I) to the coupling agent is about 1:1.2.

Embodiment 12B. The method of embodiment 7B, wherein the coupling agent is added to the reaction mixture before the amination reagent.

Embodiment 13B. The method of any one of embodiments 1B-12B, wherein the reaction temperature is between about 15° C. and 50° C. during step (i).

Embodiment 14B. The method of any one of embodiments 1B-13B, wherein step (ii) is conducted in a polar aprotic solvent.

Embodiment 15B. The method of embodiment 14, wherein the polar aprotic solvent selected from the group consisting of acetone, acetonitrile, dichloromethane, chloroform, dimethylformamide, dimethylsulfoxide, ethyl acetate, tetrahydrofuran, diethyl ether, dioxane, furan, dimethoxyethane, methyl tert-butyl ether, and chlorobenzene.

Embodiment 16B. The method of embodiment 15B, wherein the polar aprotic solvent is tetrahydrofuran.

Embodiment 17B. The method of embodiment 1, wherein the reducing conditions comprise a reducing agent.

Embodiment 18B. The method of embodiment 17B, wherein the reducing agent is hydrogen or a hydride reagent.

Embodiment 19B. The method of embodiment 18B, wherein the reducing agent is hydrogen, LiAlH₄, NaBH₄, or di-isobutyl aluminum hydride (DIBAL-H).

Embodiment 20B. The method of embodiment 19B, wherein the reducing agent is LiAlH₄.

Embodiment 21B. The method of embodiment 17B, wherein the reducing conditions further comprise a metal halide.

Embodiment 22B. The method of embodiment 21B, wherein the metal halide is AlCl₃.

Embodiment 23B. The method of embodiment 17B, wherein the reducing conditions comprise LiAlH₄ and AlCl₃ in a molar ratio of between about 4:1 and 1:4.

Embodiment 24B. The method of embodiment 23B, wherein the reducing conditions comprise LiAlH₄ and AlCl₃ in a molar ratio of about 3:1.

Embodiment 25B. The method of any one of embodiment 1B-24B, wherein the reaction temperature is between about 35° C. and 100° C. during step (ii).

Embodiment 26B. The method of embodiment 1, further comprising reacting the compound of Formula (III) with an acid to produce a compound of Formula (IV):

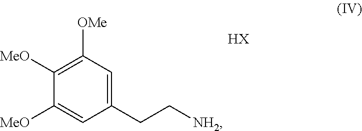

wherein X is the conjugate base of the acid.

Embodiment 27B. The method of embodiment 26B, wherein the acid is an inorganic acid.

Embodiment 28B. The method of embodiment 27B, wherein the inorganic acid is any from the group consisting of HCl, HBr, HI, HF, HNO₃, H₃PO₄, H₂SO₄, H₃BO₃, and HClO₄.

Embodiment 29B. The method of embodiment 28B, wherein the inorganic acid is HCl, HBr, HI, or H₂SO₄.

Embodiment 30B. The method of embodiment 29B, wherein the inorganic acid is HCl.

Embodiment 31B. The method of embodiment 27B, wherein the ratio of the compound of Formula (III) to the inorganic acid is about 1:1 to about 1:100.

Embodiment 32B. The method of embodiment 26B, wherein reacting the compound of Formula (III) with an acid is conducted in a solvent selected from the group consisting of acetone, methanol, ethanol, propanol, isopropanol, n-butanol, isobutanol, tetrahydrofuran, isopropyl acetate, acetonitrile, and methyl ethyl ketone.

Embodiment 33B. The method of embodiment 32B, wherein the solvent is isopropanol.

Embodiment 34B. The method of embodiment 26B, further comprising isolating the compound of Formula (IV) by filtration.

Embodiment 35B. The method of any one of embodiments 1B-34B, wherein the method is performed under Good Manufacturing Practices (GMP) conditions.

Embodiment 36B. A composition comprising a compound of Formula (II),

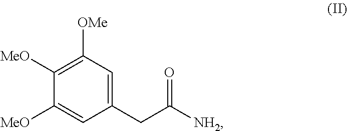 [SMILES: COc1cc(CC(N)=O)cc(OC)c1OC]

and any of:
a. between about 0.1 wt % and 5 wt % imidazole;
b. between about 0.1 wt % to 1.0 wt % dichloromethane; and
c. between about 0.1 wt % and 3.0 wt % 3,4,5-trimethoxyphenyl acetic acid.

Embodiment 37B. A composition comprising a compound of Formula (III),

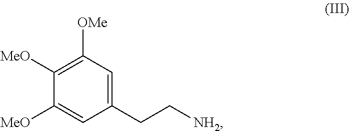 [SMILES: COc1cc(CCN)cc(OC)c1OC]

and any of:
a. between about 0.1 wt % and 8.0 wt % 2-(3,4,5-trimethoxyphenyl)acetamide;
b. between about 0.1 wt % and 5 wt % dimeric amine;
c. between about 0.1 wt % and 5 wt % n-butanol; and
d. between about 0.1 wt % and 6% toluene.

Embodiment 38B. A composition comprising a compound of Formula (IV-a),

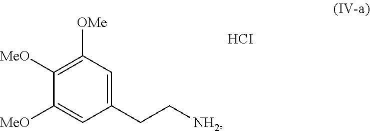 [SMILES: COc1cc(CCN)cc(OC)c1OC ; "HCI"]

and any of:
a. between about 0.1 wt % and 8.0 wt % 2-(3,4,5-trimethoxyphenyl)acetamide;
b. between about 0.01 wt % and 5 wt % 4-(2-aminoethyl)-2,6-dimethoxyphenol;
c. between about 0.01 wt % and 5 wt % 5-(2-aminoethyl)-2,3-dimethoxyphenol;
d. between about 1 ppm and 50 ppm isopropanol;
e. between about 1 ppm and 50 ppm methanol;
f. between about 500 ppm and 1000 ppm ethanol;
g. between about 0.01 wt % and 0.25 wt % water;
h. between about 10 wt % and 20 wt % chloride;
i. between about 0.1 ppm and 10 ppm Li;
j. between about 0.1 ppm and 200 ppm Al;
k. between about 0.1 wt % and 8.0 wt % 2-(3,4,5-trimethoxyphenyl)acetamide;
l. between about 0.01 wt % and 5 wt % 4-(2-aminoethyl)-2,6-dimethoxyphenol; and
m. between about 0.01 wt % and 5 wt % 5-(2-aminoethyl)-2,3-dimethoxyphenol.

Embodiment 39B. A pharmaceutical composition, comprising the composition of any one of embodiments 36B to 38B; and a pharmaceutically acceptable carrier, diluent, or excipient.

Embodiment 40B. A method of treating a substance abuse disorder, comprising administering to a subject in need thereof the composition of any one of embodiments 36B to 38B, or the pharmaceutical composition of embodiment 39B.

### EXAMPLES

The invention now being generally described, it will be more readily understood by reference to the following examples which are included merely for purposes of illustration of certain aspects and embodiments of the present invention, and are not intended to limit the invention in any way. The following synthetic schemes are provided for purposes of illustration, not limitation. The following examples illustrate the various methods of making compounds described herein. It is understood that one skilled in the art may be able to make these compounds by similar methods or by combining other methods known to one in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-5 show general and exemplary procedures for the preparation of mescaline and mescaline salts.

### Example 1—Exploratory Synthesis of Mescaline

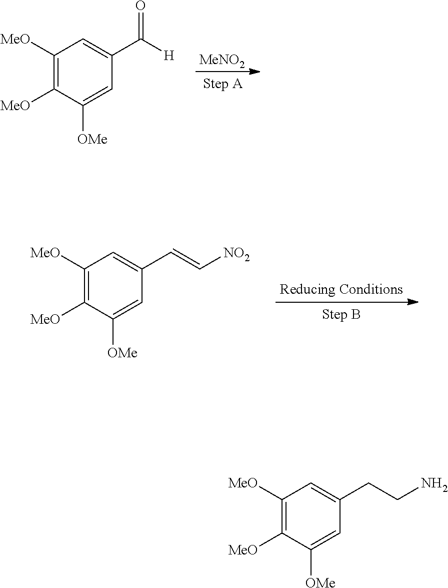 [SMILES: "MeNO2" ; COc1cc(C=O)cc(OC)c1OC ; COc1cc(/C=C/[N+](=O)[O-])cc(OC)c1OC ; COc1cc(CCN)cc(OC)c1OC]

Step A: Initial work into the synthesis of Mescaline was carried out. Trials were performed on Step A with either (i) nitromethane/NH₄OAc/AcOH or (ii) nitromethane/NaOH/MeOH. The results from these trials indicated that the conditions of (i) were more suitable for the production of the nitro-intermediate.

A series of trials varying the reaction conditions was performed as follows: Reaction volume: 2 vols, 3.5 vols (initial conditions) and 5 vols; Nitromethane amount: 1.5 eq, 2.0 eq and 3.0 eq (initial conditions); Ammonium acetate amount: 2.0 eq, 3.0 eq; Addition of nitromethane at 90° C. (using initial conditions); Higher reaction temperature (110° C.).

The reactions were stirred at 90° C., with sampling after 1, 2 and 4 hours. Based on the HPLC results obtained, the following conclusions were made: Reducing the reaction volume gave a more rapid conversion to product but with an increased level of impurities when compared to the results of the initial conditions; Increasing the reaction volume resulted in a slightly slower rate of conversion, with a slight improvement in the purity profile; Increasing the reaction volume resulted in a slightly slower rate of conversion, with a slight improvement in the purity profile; Increasing the amount of ammonium acetate increased the reaction rate significantly. However, only slightly extended stir out times (2 hours compared to 1 hour) resulted in significant product degradation; Addition of the nitromethane at 90° C. did not result in a more rapid conversion to product, with very little variation from the initial all-onboard conditions; Higher reaction temperature gave an increase in impurity formation.

Following on the results described above the conditions chosen for Stage 1 were as follows: 3.0 eq Nitromethane; 1.0 eq ammonium acetate; 3.5 vols acetic acid; 90° C. reaction temperature for 2-4 hours. These conditions were scaled up to 100 g of aldehyde, which gave 71.2 g (64% yield corrected for sampling) after the reaction and subsequent recrystallisation from EtOH, with a purity of >95% by NMR.

Step B: Initial reducing conditions were conducted using a variety of reducing conditions for the production of mescaline. These conditions were as follows: LiAlH₄; BH₃-THF; BH₃-THF/NaBH₄; Fe/HCl/MeOH.

The Fe/HCl/MeOH conditions resulted in decomposition, with the remaining conditions producing 10-20% of an amino-alcohol impurity alongside mescaline. Additional studies were done in an attempt to reduce the level of this impurity by conversion to the HCl salt and subsequent recrystallisation. This reduced the impurity to ˜1.5%, with most of the other impurities purged, however the overall yield was relatively low. Thus, new methods were explored as provided in the subsequent examples to overcome the low yield of product.

### Example 2—Synthesis of 2-(3,4,5-Trimethoxyphenyl)Acetamide

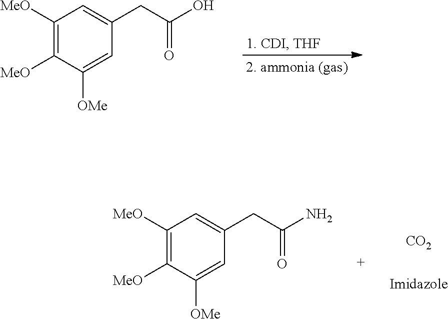 [SMILES: COc1cc(CC(=O)O)cc(OC)c1OC ; "1. CDI, THF" ; COc1cc(CC(N)=O)cc(OC)c1OC ; "CO2"]

The use of carbonyldiimidazole (CDI) was subsequently investigated, with DCM as solvent. The formation of the CDI-adduct worked well and subsequent treatment with aqueous ammonia gave a cleaner profile than that obtained from the acid chloride reaction. The use of CDI was subsequently investigated, with DCM as solvent. The formation of the CDI-adduct worked well and subsequent treatment with aqueous ammonia gave a clean profile. However, the isolated yield was low (40%) and it was found that there was significant product in the aqueous layers. The solvent was exchanged with THE in an effort to allow for more efficient removal of imidazole during work-up, however after work-up, the level was 22% w/w. The work-up was also challenging, requiring addition of significant amounts of EtOAc and brine to give two layers, with losses of product to the aqueous layer.

The conditions were further modified to address the previous deficiencies. The aqueous/THF conditions were scaled-up to 50 g of acid, using 1.2 eq of CDI in 10 volumes of THF. Both the initial CDI adduct formation and the subsequent treatment with ammonia were successful. A modified work-up procedure gave improvements, providing 75 g of a white solid that was 57% amide product by NMR (86% active yield), with the remainder being imidazole. This material was recrystallized from THE (5 vols to acid mass), to give 23 g (46% overall yield) of product with a purity of 99.9% by HPLC and >95% by NMR, with imidazole not detected.

A final modification was performed on a 25 gram scale. The formation of the CDI adduct proceeded as expected with 1.2 eq CDI in 10 volumes THF, then the reaction mixture was sparged with ammonia gas for 30 minutes (exotherm to 30° C.). Analysis by HPLC showed complete conversion to 2-(3,4,5-trimethoxyphenyl)acetamide. Unlike previous reactions a thick slurry was formed at this point. A sample of this slurry was filtered and analysis of the solids and filtrates showed product in both, thus the mixture was concentrated and the isolated product purified by THE recrystallization (3 vols to acid starting material).

Recrystallization of this product afforded 14 g (56%) of 2-(3,4,5-trimethoxyphenyl)acetamide, which contained 4 mol % imidazole by NMR. Attempts to purify 2-(3,4,5-trimethoxyphenyl)acetamide using various recrystallization solvents were conducted. However, these were unsuccessful at removing the imidazole impurity.

Base washes were used in lieu of recrystallization of the crude product to afford a higher recovery of 2-(3,4,5-trimethoxyphenyl)acetamide and lower imidazole content. The crude material (5 grams) was dissolved in DCM (10 vols) and washed multiple times with saturated aq. NaHCO₃ (5 vols). This process was repeated with the organic layers combined and dried, which afforded 3.7 grams (79% yield) of product containing 8 mol % imidazole.

In order to assess whether residual imidazole had an effect on the production of mescaline in the next step, a 3.5 g scale reaction was performed using the product that contained 8 mol % imidazole. The reaction worked as expected, with no change in the profile compared to previous batches, demonstrating that up to 8 mol % content imidazole can be tolerated for the subsequent synthetic step.

Table 1 provides the process for the production of 2-(3,4,5-trimethoxyphenyl)acetamide on a 450 gram scale. Final weight of product was 338 grams (75% yield).

TABLE 1
Synthetic Steps for the Synthesis of 2-(3,4,5-trimethylmethoxyphenyl)acetamide

| Step | Process |
|---|---|
| 1 | Add 3,4,5-trimethoxyphenyl acetic acid (1.0 g/g, limiting reagent) to the vessel |
| 2 | Add THF (10 mL/g) to the vessel. Turn on the stirrer. Stir for 15 minutes, until the |
| | majority of the solid dissolves. Set jacket to 15-25° C. |
| 3 | Add CDI (0.86 g/g, 1.20 eq) portion wise over at least 40 minutes, allowing it to |
| | dissolve before addition of the next portion. |
| 4 | On completion of addition stir at 15-25° C. for a minimum of 2 hours. |
| 5 | Set up a sparging tube into the vessel and an exhaust pipe into a carboy of water. |
| 6 | Set the jacket to 20° C. |
| 7 | Sparge the reaction with ammonia gas at a rate of ~0.5 L/min for at least 45 minutes |
| | at 20-32° |
| 8 | Cool the mixture back to 15-25° C. |
| 9 | Concentrate the mixture in vacuo |
| 10 | Add the solid back to the reaction vessel. |
| 11 | Add DCM (10 mL/g) to the vessel. Turn on the stirrer. Set the jacket to 15-25° C. |
| 12 | Add saturated aqueous NaHCO₃ (5 mL/g) over 5 min. |
| 13 | Stir the mixture for at least 30 minutes at 15-25° C. |
| 14 | Separate the layers |
| 15 | Recharge the bottom organic layer to the vessel |
| 16 | Add saturated aqueous NaHCO₃ (3 mL/g) over 5 min. |
| 17 | Stir the mixture for at least 20 minutes at 15-25° C. |
| 18 | Separate the layers |
| 19 | Recharge the bottom organic layer to the vessel |
| 20 | Add saturated aqueous NaHCO₃ (3 mL/g) over 5 min. |
| 21 | Stir the mixture for at least 20 minutes at 15-25° C. |
| 22 | Separate the layers |
| 23 | Dry over Na₂SO₄, filter and wash solids with DCM (2 mL/g) |
| 24 | Concentrate the filtrate in vacuo at 40° C. |
| 25 | Dry the solids in vacuo at 40° C. until constant mass is achieved. |

¹H NMR (CDCl₃) provided 98.257 wt % 2-(3,4,5-trimethylmethoxyphenyl)acetamide, 0.556 wt % dichloromethane, and 1.187 wt % imidazole. HPLC provided 96% purity.

### Example 3—Synthesis of Mescaline

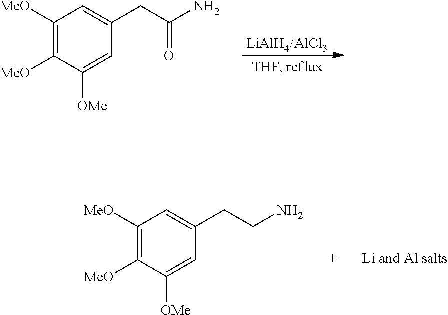 [SMILES: COc1cc(CC(N)=O)cc(OC)c1OC ; "LiAlH4/AlCl3" ; COc1cc(CCN)cc(OC)c1OC]

Table 2 provides the process or the production of Mescaline on a 325 gram scale. Final weight of product was 242 grams (79% yield).

TABLE 2
Synthetic Steps for the synthesis of Mescaline

| Step | Process |
|---|---|
| 1 | Add THF (2.62 mL/g) to a first vessel. Turn on stirrer. |
| 2 | Add AlCl₃ (0.43 g/g, 0.73 eq) portion wise over 30-60 minutes to the first vessel, keeping |
| | T <30° C., allowing the majority of the solid to dissolve between additions. |
| 3 | Stir the mixture for at least 30 minutes, allowing temperature to return to 15-25° C. |
| 4 | Add LiAlH₄ (2.4M in THF, 4.06 mL/g, 2.2 eq) to the AlCl₃ solution, dropwise over |
| | 30-60 minutes, keeping T <30° C. |
| 5 | Stir the AlCl₃/LiAlH₄ mixture at 15-25° C. for 30 minutes |
| 6 | To a second vessel add 2-(3,4,5-trimethylmethoxyphenyl)acetamide from step 1, (1 g/g, |
| | limiting reagent) |
| 7 | Add THF (5 mL/g). Turn on stirrer |
| 8 | Cool mixture to 5-15° C. |
| 9 | Add AlCl₃/LiAlH₄ mixture from Step 5, dropwise over at least 45 minutes, keeping T <30° C. |
| 10 | On completion of addition heat the mixture to 60-67° C. stir for at least 16 hours |
| 11 | Cool reaction mixture to room temperature (15-25° C.) Divide into 2 equal portions if |
| | carrying out split work-up. |
| 12 | To a clean second vessel. add methanol (0.936 mL/g) |
| 13 | Charge ethyl acetate (17.1 mL/g) |
| 14 | Charge Rochelle salts (11.5 mL/g) (53% aq); 12.2 kg Rochelle salts added to 17440 mL |
| | of water makes 23 L of 53% |
| 15 | Cool the quench mixture to between 5 and 10° C. |
| 16 | Add the reaction mixture into the quench solution (reverse addition) over at least 2 hours |
| | keeping the temp below 30° C. (Either use a dropping funnel for the addition or transfer |
| | the batch into a 20 L flange flask and use N2 push) |
| 17 | Stir the quenched reaction mixture for minimum of 15 minutes. Temp = 15-25° C. |
| 18 | Separate phases. Charge Ethyl acetate (3.95 mL/g) to assist separation. Temp = 15-25° C. |
| 19 | Charge Aq. phase back to the vessel and extract with ethyl acetate (7.36 mL/g) stir |
| | for 15 mins and separate. Charge ethyl acetate (3.94 mL/g) to assist separation. |
| | Temp = 15-25° C. |
| 20 | Charge Aq. phase back to the vessel and extract with Ethyl acetate (7.36 mL/g) stir for 15 |
| | mins and separate. Charge Ethyl acetate (3.94 mL/g) to assist separation. Temp = 15-25° C. |
| 21 | Dry combined organic extracts over sodium sulphate |
| 22 | Filter dried organics, and wash with ethyl acetate (1.5 mL/g) |
| 23 | Concentrate combined organics at 40-50° C. under vacuum. Azeotrope with toluene (2 × |
| | 1.5 mL/g). Product is an oil, stored in a duran bottle rinsed in with IPA (1 mL/g) and |
| | purge with N₂. |

NMR of the final material identified 4.2% toluene and 1.6% n-butanol as impurities in the product. HPLC of the final material identified 2-(3,4,5-trimethoxyphenyl)acetamide, dimeric amine (MW 405 g/mol), and additional oligomeric amines as impurities.

### Example 4—Synthesis of Mescaline Hydrochloride

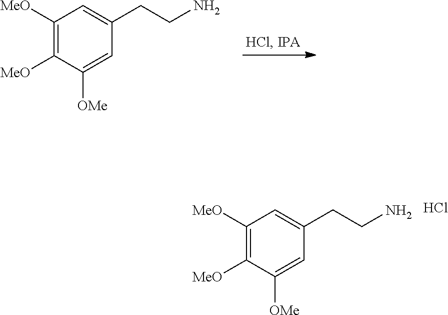 [SMILES: COc1cc(CCN)cc(OC)c1OC ; Cl ; COc1cc(CCN)cc(OC)c1OC]

Table 3 provides the process for the production of Mescaline Hydrochloride on a 233 gram scale. Final weight of product was 195 grams (71% yield).

TABLE 3
Synthetic Steps for the Synthesis of Mescaline Hydrochloride

| Step | Process |
|---|---|
| 1 | Dissolve Mescaline free base (1.0 g/g active) in IPA (4.5 mL/g) |
| 2 | Polish filter the mescaline free base solution into the vessel |
| | at 15-25° C. |
| 3 | Rinse the mescaline container with polish filtered IPA (0.5 mL/g) |
| | and use this to rinse the lines into the vessel. |
| 4 | Turn on the stirrer. Set the jacket to 10° C. |
| 5 | Polish filter the HCl in IPA into an addition funnel by nitrogen |
| | push then add to the vessel dropwise over at least 45-60 minutes, |
| | maintaining T <25° C. |
| 6 | On completion of addition cool the mixture to 0-5° C. and stir for |
| | 1 hour |
| 7 | Filter the batch |
| 8 | Add polish filtered IPA (0.5 mL/g) to the vessel and stir for 5 |
| | minutes then use to wash the filter cake. Leave to pull dry for |
| | at least 15 minutes |
| 9 | Add polish filtered IPA (0.5 mL/g) to the vessel and stir for 5 |
| | minutes then use to wash the filter cake. Leave to pull dry for |
| | at least 30 minutes. |
| 10 | Dry the solid in vacuo at 40° C. until constant weight is achieved. |

¹H NMR (d⁶-DMSO) provided 99.569 wt % Mescaline HCl and 0.404 wt % isopropanol.

### Example 5—Purification of Mescaline Hydrochloride

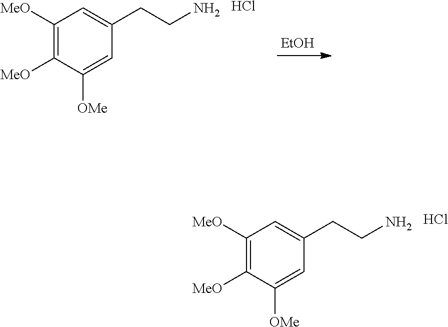 [SMILES: "EtOH" ; Cl ; COc1cc(CCN)cc(OC)c1OC ; Cl ; COc1cc(CCN)cc(OC)c1OC]

Table 4 provides the process for the purification of Mescaline Hydrochloride on a 247 gram scale. Final weight of product was 204 grams (82.5% yield).

TABLE 4
Steps For The Purification of Mescaline Hydrochloride

| Step | Process |
|---|---|
| 1 | Add Mescaline HCl salt (1.0 g/g) to the vessel |
| 2 | Polish filter EtOH (5 mL/g) to the vessel and start the stirrer |
| 3 | Heat the suspension to 77-80° C. and stir until a solution |
| | is obtained. |
| 4 | Cool the mixture to 0-5° C. over at least 1.5 hours |
| 5 | Stir at 0-5° C. for at least 30 minutes |
| 6 | Filter the mixture |
| 7 | Add polish filtered EtOH (0.5 mL/g) to the vessel and stir for 5 |
| | minutes then uset his to wash the filter cake. Allow to pull dry |
| | for at least 10 minutes. |
| 8 | Add polish filtered EtOH (0.5 mL/g) to the vessel and stir for 5 |
| | minutes then use this to wash the filter cake. Allow to pull dry |
| | for at least 30 minutes. |
| 9 | Dry the solid in vacuo at 40° C. until constant weight is achieved. |
| | A white to off-white solid is obtained. |

¹H NMR (d⁶-DMSO) provided 99.537 wt % Mescaline HCl and 0.463 wt % ethanol.

A batch analysis provided a final purity of Mescaline hydrochloride of 99.7% by HPLC; Water content by Karl Fischer 0.11 wt %; Residual solvents: 0.08% (836 ppm ethanol); Chloride content by titration: 14.74 wt %; and Elemental Analysis: Li, 0.6 ppm and Al <0.1 ppm.

### Example 6—Production Scale Synthesis of Mescaline Hydrochloride

### Step 1: Synthesis of 2-(3,4,5-trimethoxyphenyl)acetamide

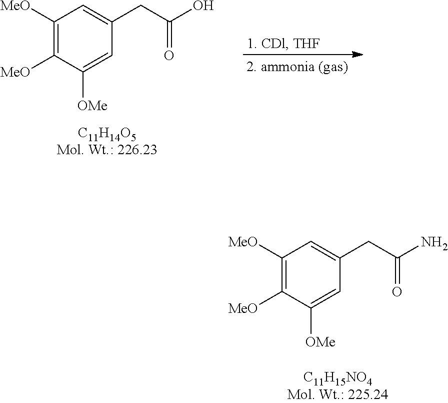 [SMILES: COc1cc(CC(=O)O)cc(OC)c1OC ; "1. CDl, THF" ; COc1cc(CC(N)=O)cc(OC)c1OC]

Four batches of 2-(3,4,5-trimethoxyphenyl)acetamide were synthesized using in a total of 11 kg of the phenyl acetic acid starting material and resulting in 8.655 kg of 2-(3,4,5-trimethoxyphenyl)acetamide (79% active yield). This yield was higher than the anticipated yield of 8256 g (75.4% active yield).

### Batch No. 1 Synthetic Procedure

To a clean 50 L vessel under nitrogen was charged 3,4,5-trimethoxyphenylacetic acid (2750 g, 12.15 mol) and THE (27.5 L, 10 vol). This was then added with carbonyl diimidazole (CDI, 2365 g, 14.58 mol) portion wise over 40 minutes, maintaining a temperature of between 15 and 25° C. The reaction mixture was then stirred between 15 and 25° C. for 2 hours. HPLC of a sample at this stage was quenched with methanol and indicated adduct formation. The reaction was then sparged with ammonia gas for 50 mins. A sample at this stage was taken and diluted with methanol, the diluted sample was subsequently analyzed via HPLC and indicated that the reaction was complete.

The reaction mixture was then concentrated under vacuum at 40° C. on a rotary evaporator to produce an off white solid. DCM (4 L, 1.45 vol) was charged to the Buchi flask at 40° C. to mostly dissolve the solids. The resulting fine suspension was charged to the 50 L vessel with DCM (23.5 L, 8.54 vol). The reaction mixture was cooled to between 15 and 25° C. and saturated aqueous sodium hydrogen carbonate (13.75 L, 5 vol) was charged, stirred for 30 minutes and the layers were separated. The organic phase was charged back to the vessel and washed twice with saturated sodium hydrogen carbonate (2×8250 mL) and separated. The organic phase was dried over sodium sulphate, filtered, and concentrated under vacuum at 40° C. to give an off white solid. The subsequent wet solid was dried under vacuum in an oven for 16 hours at 40° C. to produce 2-(3,4,5-trimethoxyphenyl)acetamide (2252 g, 82% yield). ¹H NMR analysis indicated >95% purity with <2% imidazole remaining. HPLC analysis showed purity of 98.7%. Karl-fisher (KF) analysis showed <0.3% water present. Advantageously, this product containing imidazole was used in the subsequent production step without the need of additional work-up procedures. Analogous procedures were conducted for batches 2-4, with the results of each batch in Table 5.

TABLE 5
Production Overview of Batches 1-4

| Batch | Input | Output | Yield | ¹H NMR | HPLC Purity | |
|---|---|---|---|---|---|---|
| No. | Mass (g) | Mass (g) | (%) | purity (%) | (% a/a) | KF |
| 1 | 2750 | 2251 | 82 | >95% | 98.3% | 0.3% |
| 2 | 2750 | 2211 | 80 | >95% | 99.3% | 0.43% |
| 3 | 2750 | 2142 | 78 | >95% | 99% | 0.17% |
| 4 | 2750 | 2051 | 75 | >95% | 98.6% | 0.33% |
| Total | 11000 | 8655 | | | | |

### Step 2: Synthesis of Mescaline

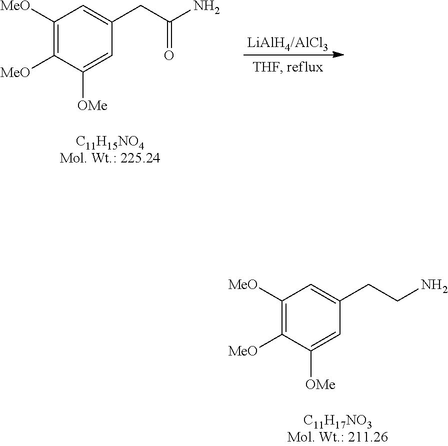 [SMILES: COc1cc(CC(N)=O)cc(OC)c1OC ; "LiAlH4/AlCl3" ; COc1cc(CCN)cc(OC)c1OC]

Work up Parameters: Initial synthesis of mescaline was conducted at a 530 g scale due to gelation upon a sodium hydroxide quench. Upon scale up, similar gelation was observed indicating that the process was not suitable for 50 L scale up. The material from this batch was processed to afford 338 g of mescaline (Trial batch, 64% against a target of 79.5% yield).

Due to the low yield and a workup that was not suitable for 50 L processing, development of an alternative quench and work up was required. A 50 L batch was performed processing 2000 g of 2-(3,4,5-trimethoxyphenyl)acetamide to produce reasonable amounts of mescaline to attempt various work ups. HPLC analysis of the large-scale batch showed 86.83% product which met the desired completion specification.

From the bulk reaction mixture two 1 L portions were taken through a methanol, ethyl acetate and Rochelle salts work up. Two work procedures were used to evaluate ideal parameters for the quench. In work up 1 the reaction mixture was added with the quench solvents, whilst in work up 2 a reverse quench was performed using Batch No. 5.

Work up 1: Afforded 53 g active mescaline (66% active yield). HPLC analysis showed 83% purity with a 5% impurity which was shown to be the acetamide (structure below):

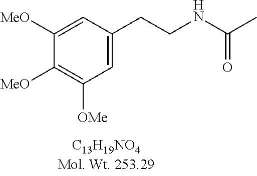 [SMILES: COc1cc(CCNC(C)=O)cc(OC)c1OC]

Work up 2 (reverse quench): Afforded 69 g active mescaline (84% active yield) HPLC analysis showed a 90% purity with none of the acetamide present.

The reverse quench was shown to be better producing a higher yield and purity by HPLC. This procedure was scaled up to 5 L. A total of 348.3 g active mescaline was isolated. HPLC analysis showed 90% purity with none of the acetamide impurity. This procedure was scaled up to process the remaining 16 L of reaction mixture from Batch No. 6 which afforded 1188 g active mescaline (91.5% active yield based on NMR). HPLC showed 84.3% purity with 4.8% of the acetamide impurity previously seen in the 1 L forward quench. Thus, extended processing times on scale up cause the acetamide to form at varying levels. The acetamide is completely purged during the salt formation and purification stages and therefore only causes a modest loss in yield.

### Batch No. 7 Synthetic Procedure

To a clean 20 L vessel under nitrogen was charged THE (5.24 L, 2.62 vol). This was followed by the addition of aluminum trichloride (860 g, 8.58 mol) portion wise over 60 minutes, maintaining temperature <30° C. LiAlH₄ (8.12 L, 2.4M) was then charged dropwise over 80 minutes maintaining a temperature <30° C., a pale grey solution formed. The mixture was cooled to between 15 and 25° C. and stirred for 30 minutes. To a separate 50 L vessel under nitrogen was charged mescaline stage 1 (2000 g, 8.87 mol) and THE (10 L, 5 vol) and the resulting suspension cooled to between 5 and 15° C. To this cooled suspension the LiAlH₄/AlCl₃ mixture was charged dropwise over 1 hour maintaining temperature <35° C. (exotherm reached 32° C.). The resulting solution was heated to 67° C. and stirred. A sample was taken and analyzed using HPLC which confirmed completion and showed 86.9% product with 0.6% starting material remaining.

The batch was then cooled to room temperature and separated into two portions to perform a split batch work up. For each portion of the reaction mixture (11.5 L) the following procedure was used. The reaction mixture (11.5 L) was charged to a clean 20 L vessel under nitrogen. To the 50 L vessel, methanol (1.3 L, 0.65 vol), ethyl acetate (17.1 L 17.1 vol) and Rochelle salts (aq 53%) (11.5 L, 5.75 vol) were charged and cooled to between 0 and 5° C. The reaction mixture was then charged dropwise via a dip pipe over 2 hours maintaining temperature <30° C. (exotherm reached 26° C.). Once quenched the mixture was stirred for 15 minutes at room temperature and then allowed to separate, ethyl acetate (5.5 L) was charged to aid separation. The resulting aqueous layer was back extracted with ethyl acetate (2×7.36 L). The combined organic phases were dried over sodium sulphate, filtered, and concentrated under vacuum in a rotary evaporator at 50° C. and azeotroped with toluene (3×3 L). This produced 1620 g active mescaline as a pale brown oil (87% active yield based on NMR). HPLC showed a purity of 86.4% with 3% of the acetamide impurity. Batch Nos. 7-8 were similarly processed and the results of each batch are summarized below in Table 6.

TABLE 6
Production Overview of Trial batch and Batches 5-8

| | | | | ¹H NMR | | |
|---|---|---|---|---|---|---|
| | | Output | Acetamide | (excluding | HPLC (% | Yield |
| Batch | Input (g) | (g) | (% a/a) | solvents) | a/a) | (%) |
| Trial | 530 | 338 | 0 | >90% | 89.75% | 68% |
| 5 | 2000 | 1653 | 4.82 | >90% | 84.29% | 88% |
| 6 | 2000 | 1620 | 3.00 | >90% | 86.47% | 87% |
| 7 | 2100 | 1663 | 2.90 | >90% | 82.8% | 85% |
| 8 | 2095 | 1819 | 2.57 | >90% | 86.2% | 93% |
| Total | 8725 | 7093 | | | | |

### Step 3: Synthesis of Mescaline Hydrochloride

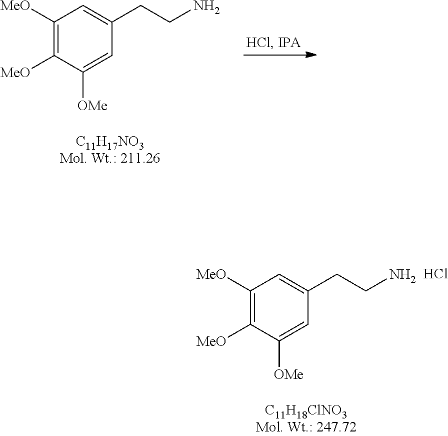

The synthesis of mescaline hydrochloride was split over two batches using 7064 g of mescaline. This resulted in 6390 g mescaline hydrochloride in 76.9% active yield following the procedure previously developed. This stage proceeded as expected and successfully provided more than the target quantity of mescaline hydrochloride (5.07 kg). The amide was reduced to <0.5% in this process.

### Batch No. 10 Synthetic Procedure

Mescaline (5183 g, 24.53 mol) was dissolved in IPA (17.247 L, 3.32 vol) and charged to a clean 50 L under nitrogen, IPA (400 mL, 0.07 vol) was used as a line rinse. The mixture was cooled to between 15-20° C. (17.2° C. actual) and HCl in IPA (4629 mL, 5.3M) was charged dropwise over 60 mins maintaining the temperature <25° C. The reaction mixture was then cooled to 0-5° C. and held for 1 hour, after which it was filtered and washed with IPA (2×2591.5 ml, 2×0.5 vol). The resulting mescaline hydrochloride was oven dried at 40° C. for 2 days to afford 4761 g (78.3% yield). ¹H NMR indicated a purity >95% with 0.7% w/w IPA present. HPLC indicated a 94.7% chemical purity with a total of 2.28% of the demethylated species present at RRT 0.72 and RRT 0.77 respectively (structures shown below) and 0.42% of the acetamide at RRT 1.32. A similar procedure was used for Batch No. 9 and the results of each batch are summarized below in Table 7.

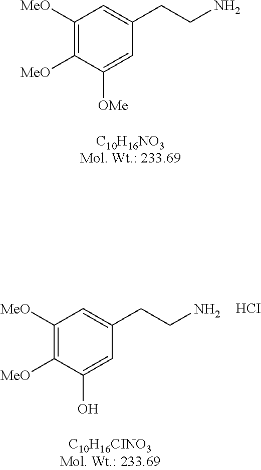

TABLE 7
Production Overview of Batches 9-10

| | | | | | | Demethylated | | |
|---|---|---|---|---|---|---|---|---|
| | | | | Purity by | | Species by | | Acetamide |
| | | | | ¹H NMR | Purity by | HPLC (% a/a) | | by HPLC |
| | Input | Output | Yield | (excluding | HPLC | RRT | RRT | (% a/a) |
| Batch | (g) | (g) | (%) | solvents) | (%) a/a | 0.72 | 0.77 | RRT 1.31 |
| 9 | 1881 | 1629 | 73.8 | >95% | 94.3% | 0.99 | 0.80 | 0.45% |
| 10 | 5183 | 4761 | 78.3 | >95% | 94.7% | 1.16 | 1.48 | 0.42% |
| Total | 7064 | 6390 | | | | | | |

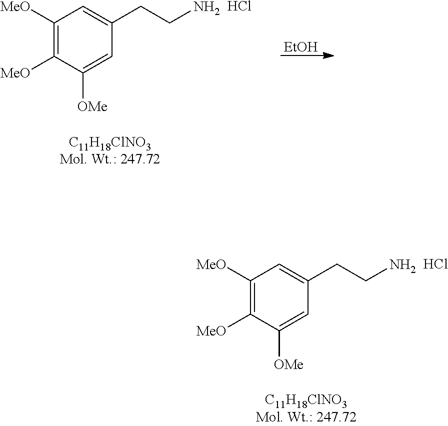 [SMILES: "EtOH" ; Cl ; COc1cc(CCN)cc(OC)c1OC ; Cl ; COc1cc(CCN)cc(OC)c1OC]

The purification of mescaline hydrochloride was split over two batches using 1200 g and 5196 g respectively. This afforded a total of 5324 g mescaline hydrochloride (83.2% active yield) against a target of 4 kg. NMR of both batches indicated a purity of >95% and LC indicated a purity of ≥99%.

### Batch No. 12 Purification Procedure

To a clean 50 L vessel under nitrogen, mescaline. HCl (5196.5 g, 20.97 mol) and ethanol (25.9 L, 5 vol) were charged. The resulting suspension was heated to 77-80° C. until dissolution was achieved (77° C. actual). The reaction was then cooled to 0-5° C. over 2 hours (product precipitates at 65° C.) and held for 30 mins. The batch was then filtered and washed with cooled ethanol (2×2598.2 mL, 2×0.5 vol). The resulting solid was oven dried at 40° C. for 2 days to afford 4360 g (84% yield). A similar procedure was used for Batch No. 11 and the results of each batch are summarized below in Table 8.

TABLE 8
Production Overview of Batches 11-12

| | Input | Output | H NMR | HPLC | Yield |
|---|---|---|---|---|---|
| Batch | (g) | (g) | (excluding solvents) | (% a/a) | (%) |
| 11 | 1200 | 964 | >95% | 99.4% | 80.3 |
| 12 | 5196 | 4360 | >95% | 99.4% | 83.9 |
| Total | 6396 | 5324 | | | |

Batch No. 11 analysis provided a final purity of Mescaline hydrochloride of 99.4% by HPLC; Water content by Karl Fischer 0.3 wt %; Residual solvents: methanol 20 ppm, ethanol 691 ppm, propan-2-ol 3 ppm, THE 7 ppm, toluene 1 ppm; Chloride content by titration: 13.1 wt %; and Elemental Analysis: Li, 3.6 ppm and Al, 139.5 ppm.

Batch No. 12 analysis provided a final purity of Mescaline hydrochloride of 99.4% by HPLC; Water content by Karl Fischer 0.2 wt %; Residual solvents: methanol 12 ppm, ethanol 689 ppm, propan-2-ol 1 ppm; Chloride content by titration: 14.7 wt %; and Elemental Analysis: Li, 0.8 ppm and Al, 5.1 ppm.

The invention claimed is:

1. A method of preparing a compound of Formula (III): 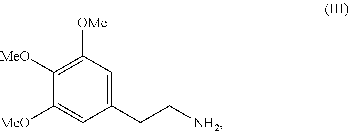 [SMILES: COc1cc(CCN)cc(OC)c1OC]
or a salt thereof, comprising:
(i) subjecting a compound of Formula (I): 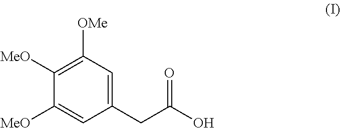 [SMILES: COc1cc(CC(=O)O)cc(OC)c1OC]
to amination conditions to form a compound of Formula (II): 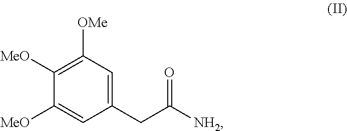 [SMILES: COc1cc(CC(N)=O)cc(OC)c1OC]
and
(ii) subjecting the compound of Formula (II) to reducing conditions to produce the compound of Formula (III), or a salt thereof.

2. The method of claim 1, wherein step (i) is conducted in a polar aprotic solvent.

3. The method of claim 2, wherein the polar aprotic solvent selected from the group consisting of acetone, acetonitrile, dichloromethane, chloroform, dimethylformamide, dimethylsulfoxide, ethyl acetate, tetrahydrofuran, diethyl ether, dioxane, furan, dimethoxyethane, methyl tert-butyl ether, and chlorobenzene.

4. The method of claim 3, wherein the polar aprotic solvent is tetrahydrofuran.

5. The method of claim 1, wherein the amination conditions comprise an amination reagent.

6. The method of claim 5, wherein the amination reagent is ammonia.

7. The method of claim 5, further comprising a coupling agent.

8. The method of claim 7, wherein the coupling agent is selected from the group consisting of dicyclohexylcarbodiimide (DCC), diisopropylcarbodiimide (DIC), 1-ethyl-3-(3-dimethylaminopropyl)carbodiimide (EDC), benzotriazol-1-yloxytris(dimethylamino)phosphonium hexafluorophosphate (BOP), 1-[bis(dimethylamino)methylene]-1H-1,2,3-triazolo[4,5-b]pyridinium 3-oxide hexafluorophosphate (HATU), carbonyldiimidazole (CDI), and carbonylditriazole.

9. The method of claim 8, wherein the coupling agent is carbonyldiimidazole (CDI).

10. The method of claim 7, wherein the molar ratio of the compound of Formula (I) to the coupling agent is between about 1:1 and 1:5.

11. The method of claim 10, wherein the molar ratio of the compound of Formula (I) to the coupling agent is about 1:1.2.

12. The method of claim 7, wherein the coupling agent is added to the reaction mixture before the amination reagent.

13. The method of any one of claims 1-12, wherein the reaction temperature is between about 15° C. and 50° C. during step (i).

14. The method of any one of claims 1-13, wherein step (ii) is conducted in a polar aprotic solvent.

15. The method of claim 14, wherein the polar aprotic solvent selected from the group consisting of acetone, acetonitrile, dichloromethane, chloroform, dimethylformamide, dimethylsulfoxide, ethyl acetate, tetrahydrofuran, diethyl ether, dioxane, furan, dimethoxyethane, methyl tert-butyl ether, and chlorobenzene.

16. The method of claim 15, wherein the polar aprotic solvent is tetrahydrofuran.

17. The method of claim 1, wherein the reducing conditions comprise a reducing agent.

18. The method of claim 17, wherein the reducing agent is hydrogen or a hydride reagent.

19. The method of claim 18, wherein the reducing agent is hydrogen, LiAlH₄, NaBH₄, or di-isobutyl aluminum hydride (DIBAL-H).

20. The method of claim 19, wherein the reducing agent is LiAlH₄.

21. The method of claim 17, wherein the reducing conditions further comprise a metal halide.

22. The method of claim 21, wherein the metal halide is AlCl₃.

23. The method of claim 17, wherein the reducing conditions comprise LiAlH₄ and AlCl₃ in a molar ratio of between about 4:1 and 1:4.

24. The method of claim 23, wherein the reducing conditions comprise LiAlH₄ and AlCl₃ in a molar ratio of about 3:1.

25. The method of any one of claims 1-24, wherein the reaction temperature is between about 35° C. and 100° C. during step (ii).

26. The method of claim 1, further comprising reacting the compound of Formula (III) with an acid to produce a compound of Formula (IV): 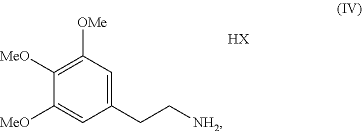
wherein X is the conjugate base of the acid.

27. The method of claim 26, wherein the acid is an inorganic acid.

28. The method of claim 27, wherein the inorganic acid is selected from the group consisting of HCl, HBr, HI, HF, HNO₃, H₃PO₄, H₂SO₄, H₃BO₃, and HClO₄.

29. The method of claim 28, wherein the inorganic acid is HCl, HBr, HI, or H₂SO₄.

30. The method of claim 29, wherein the inorganic acid is HCl.

31. The method of claim 27, wherein the ratio of the compound of Formula (III) to the inorganic acid is about 1:1 to about 1:100.

32. The method of claim 26, wherein reacting the compound of Formula (III) with an acid is conducted in a solvent selected from the group consisting of acetone, methanol, ethanol, propanol, isopropanol, n-butanol, isobutanol, tetrahydrofuran, isopropyl acetate, acetonitrile, and methyl ethyl ketone.

33. The method of claim 32, wherein the solvent is isopropanol.

34. The method of claim 26, further comprising isolating the compound of Formula (IV) by filtration.

35. The method of any one of claims 1-34, wherein the method is performed under Good Manufacturing Practices (GMP) conditions.

36. A composition comprising a compound of Formula (II), 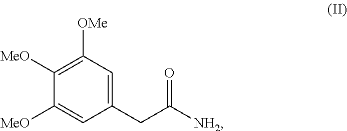 [SMILES: COc1cc(CC(N)=O)cc(OC)c1OC]
and any of:
i. between about 0.1 wt % and 5 wt % imidazole;
ii. between about 0.1 wt % to 1.0 wt % dichloromethane; and
iii. between about 0.1 wt % and 3.0 wt % 3,4,5-trimethoxyphenyl acetic acid.

37. A composition comprising a compound of Formula (III), 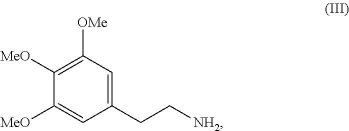 [SMILES: COc1cc(CCN)cc(OC)c1OC]
and any of:
i. between about 0.1 wt % and 8.0 wt % 2-(3,4,5-trimethoxyphenyl)acetamide;
ii. between about 0.1 wt % and 5 wt % dimeric amine;
iii. between about 0.1 wt % and 5 wt % n-butanol; and
iv. between about 0.1 wt % and 6% toluene.

38. A composition comprising a compound of Formula (IV-a), 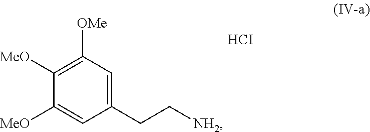 [SMILES: COc1cc(CCN)cc(OC)c1OC ; "HCI"]
and any of:
i. between about 0.1 wt % and 8.0 wt % 2-(3,4,5-trimethoxyphenyl)acetamide;
ii. between about 0.01 wt % and 5 wt % 4-(2-aminoethyl)-2,6-dimethoxyphenol;
iii. between about 0.01 wt % and 5 wt % 5-(2-aminoethyl)-2,3-dimethoxyphenol;
iv. between about 1 ppm and 50 ppm isopropanol;
v. between about 1 ppm and 50 ppm methanol;
vi. between about 500 ppm and 1000 ppm ethanol;
vii. between about 0.01 wt % and 0.25 wt % water;
viii. between about 10 wt % and 20 wt % chloride;
ix. between about 0.1 ppm and 10 ppm Li;
x. between about 0.1 ppm and 200 ppm Al;
xi. between about 0.1 wt % and 8.0 wt % 2-(3,4,5-trimethoxyphenyl)acetamide;
xii. between about 0.01 wt % and 5 wt % 4-(2-aminoethyl)-2,6-dimethoxyphenol; and
xiii. between about 0.01 wt % and 5 wt % 5-(2-aminoethyl)-2,3-dimethoxyphenol.

39. A pharmaceutical composition, comprising the composition of any one of claims 36 to 38; and a pharmaceutically acceptable carrier, diluent, or excipient.

40. A method of treating a substance abuse disorder, comprising administering to a subject in need thereof the composition of any one of claims 36 to 38, or the pharmaceutical composition of claim 39.
